(19) United States
(12) Patent Application Publication
(10) Pub. No.: US 2005/0038078 A1
(43) Pub. Date: Feb. 17, 2005

(54) Antiproliferative 2-(heteroaryl)-aminothiazole compounds and pharmaceutical compositions, and methods for their use

(75) Inventors: Wesley Kwan Mung Chong, Encinitas, CA (US); Rohit Kumar Duvadie, San Diego, CA (US); Lin Li, San Diego, CA (US); Yi Yang, San Diego, CA (US)

Correspondence Address: AGOURON PHARMACEUTICALS, INC., 10350 NORTH TORREY PINES ROAD, LA JOLLA, CA, 92037, US

(73) Assignee: Agouron Pharmaceuticals, Inc.

(21) Appl. No.: 10/639,219

(22) Filed: Aug. 11, 2003

(51) Int. Cl. A61K 31/4439; C07D 417/02

(52) U.S. Cl. 514/340; 546/270.7

(57) ABSTRACT

Compounds represented by the Formula (I): 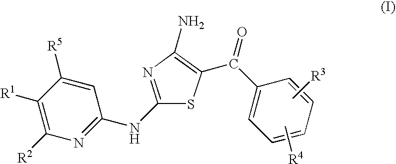 are described. The compounds and pharmaceutical compositions containing them may be used in inhibiting and/or modulating protein kinases, in treating or preventing diseases associated with protein kinases, and/or in treating or preventing cellular proliferative diseases.

### FIELD OF THE INVENTION

This invention generally relates to 2-(heteroaryl)-aminothiazole compounds and compositions containing those compounds. The invention also relates to the use of 2-(heteroaryl)-aminothiazole compounds and compositions in methods for treating and/or preventing various diseases and disorders associated with uncontrolled or unwanted cell proliferation and/or for inhibiting and/or modulating protein kinases.

### BACKGROUND OF THE INVENTION

Cell proliferation occurs in response to various stimuli and may stem from de-regulation of the cell division cycle (or cell cycle), the process by which cells multiply and divide. Hyperproliferative disease states, including cancer, are characterized by cells rampantly winding through the cell cycle with uncontrolled vigor due to, for example, damage to the genes that directly or indirectly regulate progression through the cycle. Thus, agents that modulate the cell cycle, and thus hyperproliferation, could be used to treat various disease states associated with uncontrolled or unwanted cell proliferation. In addition to cancer chemotherapeutic agents, cell cycle inhibitors are also proposed as antiparasitics (See, Gray, N.; Détivaud, L.; Doerig, C.; Meijer, L. Curr. Med. Chem. 1999, 6, 859-875) and recently demonstrated as potential antivirals (See, Schang, L. M.; Rosenberg, A.; Schaffer, P. A. J. Virol. 2000, 74, 2107-2120, Yang, Z.; Zhu, Q.; Luo, K.; Zhou, Q. Nature (London) 2001, 414, 317-322, and Nguyen, V. T.; Kiss, T.; Michels, A. A.; Bensaude, O. Nature (London) 2001, 414, 322-325). Moreover, the applicability of antiproliferative agents may be expanded to treating cardiovascular maladies such as artherosclerosis or restenosis (See Braun-Dullaeus, R. C.; Mann, M. J.; Dzau, V. J. Circulation 1998, 98, 82-89 and Fishbein, I.; Chomy, M.; Golomb, G. Drug Dev. Res. 2000, 50, 487-496), and states of inflammation, such as psoriasis and arthritis (See, Taniguchi, K.; Kohsaka, H.; Inoue, N.; Terada, Y.; Ito, H.; Hirokawa, K.; Miyasaka, N. Nat. Med. 1999, 5, 760-767).

Mechanisms of cell proliferation are under active investigation at cellular and molecular levels. At the cellular level, de-regulation of signaling pathways, loss of cell cycle controls, unbridled angiogenesis or stimulation of inflammatory pathways are under scrutiny, while at the molecular level, these processes are modulated by various proteins,

among which protein kinases are prominent suspects. Overall abatement of proliferation may also result from programmed cell death, or apoptosis, which is also regulated via multiple pathways, some involving proteolytic enzyme proteins.

Among the candidate regulatory proteins, protein kinases are a family of enzymes that catalyze phosphorylation of the hydroxyl group of specific tyrosine, serine or threonine residues in proteins. Typically, such phosphorylation dramatically perturbs the function of the protein, and thus protein kinases are pivotal in the regulation of a wide variety of cellular processes.

For example, without wishing to be bound to a particular theory, it is believed that as inhibitors of protein kinases, such as, for example, cyclin dependent kinases (“CDK”), the inventive agents can modulate the level of cellular RNA and DNA synthesis and therefore are expected to be useful in the treatment of viral infections such as HIV, human papilloma virus, herpesvirus, Epstein-Barr virus, adenovirus, Sindbis virus, poxvirus and the like. (See Schang, L. M.; Rosenberg, A.; Schaffer, P. A. J. Virol. 2000, 74, 2107-2120). Additionally, CDK5 has been implicated in the phosphorylation of tau protein, suggesting potential methods of treating or preventing Alzheimer's disease (Hosoi, T.; Uchiyama, M.; Okumura, E.; Saito, T.; Ishiguro, K.; Uchida, T.; Okuyama, A.; Kishimoto, T.; Hisanaga, S. J. Biochem. (Tokyo), 1995, 117, 741-749).

CDKs are serine-threonine protein kinases that play critical roles in regulating the transitions between different phases of the cell-cycle, such as the progression from a quiescent stage in G₁ (the gap between mitosis and the onset of DNA replication for a new round of cell division) to S (the period of active DNA synthesis), or the progression from G₂ to M phase, in which active mitosis and cell-division occurs. (See, e.g., the articles compiled in Nasmyth, K. Science 1996, 274, 1643-1645, Sherr, C. J. Science 1996, 274, 1672-1677; and Coleman, K. G.; Lyssikatos, J. P.; Yang, B. V. In Annu. Rep. Med. Chem. 1997; vol. 32, p 171-179). CDK complexes are formed through association of a regulatory cyclin subunit (e.g., cyclin A, B1, B2, D1, D2, D3, and E) and a catalytic kinase subunit (e.g., CDK1, CDK2, CDK4, CDK5, and CDK6). As the name implies, the CDKs display an absolute dependence on the cyclin subunit in order to phosphorylate their target substrates, and different kinase/cyclin pairs function to regulate progression through specific phases of the cell-cycle.

The D cyclins are sensitive to extracellular growth signals and become activated in response to mitogens during the G₁ phase of the cell cycle. CDK4/cyclin D plays an important role in cell cycle progression by phosphorylating, and thereby inactivating, the retinoblastoma protein (Rb). Hypophosphorylated Rb binds to a family of transcriptional regulators, but upon hyperphosphorylation of Rb by CDK4/cyclin D, these transcription factors are released to activate genes whose products are responsible for S phase progression. Rb phosphorylation and inactivation by CDK4/cyclin D permit passage of the cell beyond the restriction point of the G₁ phase, whereupon sensitivity to extracellular growth or inhibitory signals is lost and the cell is committed to cell division. During late G₁, Rb is also phosphorylated and inactivated by CDK2/cyclin E, and recent evidence indicates that CDK2/cyclin E can also regulate progression into S phase through a parallel pathway that is independent of Rb phosphorylation.

The progression from G₁ to S phase, accomplished by the action of CDK4/cyclin D and CDK2/cyclin E, is subject to a variety of growth regulatory mechanisms, both negative and positive. Growth stimuli, such as mitogens, cause increased synthesis of cyclin D1 and thus increased functional CDK4. By contrast, cell growth can be “reigned in,” in response to DNA damage or negative growth stimuli, by the induction of endogenous inhibitory proteins. These naturally occurring protein inhibitors include p21^{WAF1/CIP1}, p27^{KIP1}, and the p16^{INK4} family, the latter of which inhibit CDK4 exclusively (see Harper, J. W., Cancer Surv., 1997, 29, 91-107). Aberrations in this control system, particularly those that affect the function of CDK4 and CDK2, are implicated in the advancement of cells to the highly proliferative state characteristic of malignancies, such as familial melanomas, esophageal carcinomas, and pancreatic cancers (see, e.g., Hall, M.; Peters, G., Adv. Cancer Res., 1996, 68, 67-108; and Kamb, A.; et al., Science, 1994, 264, 436-440). Over-expression of cyclin D1 is linked to esophageal, breast, and squamous cell carcinomas (see, e.g., Delsal, G.; Loda, M.; Pagano, M., Crit. Rev. Oncog., 1996, 71,. 127-142). Genes encoding the CDK4-specific inhibitors of the p16 family frequently have deletions and mutations in familial melanoma, gliomas, leukemias, sarcomas, and pancreatic, non-small cell lung, and head and neck carcinomas (see Nobori, T.; et al., Nature, 1994, 368, 753-756). Amplification and/or overexpression of cyclin E has also been observed in a wide variety of solid tumors, and elevated cyclin E levels have been correlated with poor prognosis. In addition, the cellular levels of the CDK inhibitor p27, which acts as both a substrate and inhibitor of CDK2/cyclin E, are abnormally low in breast, colon, and prostate cancers, and the expression levels of p27 are inversely correlated with the stage of disease (see Loda, M.; Cukor, B.; Tam, S. W.; Lavin, P.; Fiorentino, M. et al., Nat Med. (N.Y.) 1997, 3, 231-234.). The p21 proteins also appear to transmit the p53 tumor-suppression signal to the CDKs; thus, the mutation of p53 in approximately 50% of all human cancers may indirectly result in deregulation of CDK activity. A large number of small molecule ATP-site antagonists have been identified as CDK inhibitors. (See, Webster, K. R. Exp. Opin. Invest. Drugs, 1998, 7, 865-887; Stover, D. R.; Lydon, N. B.; Nunes, J. J. Curr. Opin. Drug Disc. Dev., 1999, 2, 274-285; Gray N.; Détivaud, L.; Doerig, C.; Meijer, L. Curr. Med. Chem., 1999, 6, 859-875; Sielecki, T. M.; Boylan, J. F.; Benfield, P. A.; Trainor, G. L. In J. Med. Chem. 2000; 43, 1-18.; Crews, C. M.; Mohan, R. Curr. Opin. Chem. Biol. 2000, 4, 47-53.; Buolamwini, J. K. Curr. Pharm. Des. 2000, 6, 379-392; Rosania, G. R.; Chang, Y.-T. Exp. Opin. Ther. Patents 2000, 10, 215-230, Toogood, P. L. Med. Res. Rev. 2001, 21, 487-498, and Kimball, S. D.; Webster, K. R. Ann. Rep. Med. Chem. 2001, 36, 139-148.

In addition to the protein kinases identified above, many other protein kinases have been considered to be therapeutic targets, and numerous publications disclose inhibitors of kinase activity, as reviewed in the following: McMahon, G.; Sun, L.; Liang, C.; Tang, C. Curr. Opin. Drug Discovery Dev. 1998, 1, 131-146; Strawn, L. M.; Shawver, L. K. Expert Opin. Invest. Drugs 1998, 7, 553-573; Adams, J. L.; Lee, D. Curr. Opin. Drug Discovery Dev. 1999, 2, 96-109; Toledo, L. M.; Lydon, N. B.; Elbaum, D. Curr. Med. Chem. 1999, 6, 775-805; Garcia-Echeverria, C.; Traxler, P.; Evans, D. B. Med. Res. Rev. 2000, 20, 28-57, and Rao, R. N.; Patel, B. K. R. in Targets Cancer Chemothe. 2002, 145-178.

There is still a need, however, for more potent inhibitors of protein kinases. Moreover, as is understood by those skilled in the art, it is desirable for kinase inhibitors to possess both high affinity for the target kinase as well as high selectivity versus other protein kinases.

Among others, the following patent publications disclose thiazole compounds: WIPO International Publication Nos. WO 99/21845 and WO00/75120 disclose 2,4-diaminothiazoles used as CDK or kinase inhibitors respectively. Very recently, Roche disclosed diaminothiazoles in WIPO International Publication No. WO 02/57261. After an early report of 2,4-diaminothiazoles in Gewald, K.; Blauschmidt, P.; Mayer, R. J. Prakt. Chem. 1967, 35, 97-104, subsequent modified preparations prior to the patents above were seen in Rajasekharan, K. N.; Nair, K. P.; Jenardanan, G. C. Synthesis 1986, 353-355, Jenardanan, G. C.; Francis, M.; Deepa, S.; Rajasekharan, K. N. Syn. Comm. 1997, 27, 3457-3462, and Binu, R.; Thomas, K. K.; Jenardanan, G. C.; Rajasekharan, K. N. Org. Prep. Proced. Intl. 1998, 30, 93-96. Yet another extension of the methodology recently appeared in Devi, S. K. C.; Rajasekharan, K. N. Syn. Comm. 2002, 32, 1523-1528, which alluded to the preparation of a combinatorial library of 2,4-diaminothiazoles. This was realized from another recent modification from Masquelin, T.; Obrecht, D. Tetrahedron 2001, 57, 153-156, which was adapted to solid support in Baer, R.; Masquelin, T. J. Comb. Chem. 2001, 3, 16-19. WIPO International Publication No. WO 99/62890 discloses isothiazoles used as anticancer agents; WO 98/04536 describes thiazoles used as protein kinase C inhibitors; EP 816362A (1998) discloses thiazoles used principally for dopamine D4 receptor antagonists. Aminothiazoles were reported in U.S. Pat. No. 6,262,096, WIPO International Publication Nos. WO 01/44241, WO 01/44242, and aminobenzothiazoles in WO 99/24035. WIPO International Publication No. WO 00/17175 describes other aminothiazoles used as p38 mitogen-activated protein (MAP) kinase inhibitors, and WO 00/26202, WO 00/26203, and U.S. Pat. No. 6,114,365 describe aminothiazoles and ureidothiazoles used as anti-tumor agents. WIPO International Publication No. WO9921845 and U.S. patent application Ser. No. 10/190,219 describe aminothiazole benzamide derivatives with anti-proliferative activity. The present invention however is based on the discovery that compounds with a single nitrogen replacement in the benzamidyl ring can be surprisingly more potent than the corresponding aminothiazole compounds without the heteroatom replacement. Thus, the inventive compounds show generally more potent cell growth inhibition than the compounds described WIPO International Publication No. WO9921845 and U.S. Provisional Application Nos. 60/303,679 and 60/305,274.

### SUMMARY OF THE INVENTION

In one general aspect, the invention is directed to compounds represented by the following Formula (I): 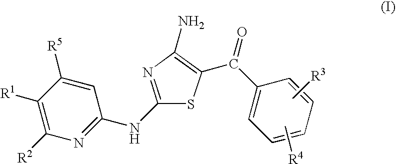
wherein:
R¹ is hydrogen, or an alkenyl, alkynyl, C₁-C₈ alkylamino, aryl, heteroaryl, cycloalkyl, heterocycloalkyl, carboxamide, sulfonamide or alkoxy group, unsubstituted or substituted with one or more substituents independently selected from the group consisting of alkyl, heteroalkyl, haloalkyl, haloaryl, halocycloalkyl, haloheterocycloalkyl, aryl, cycloalkyl, heterocycloalkyl, heteroaryl, —NO₂, —NH₂, —N—(R_{c})OR_{d}, —CN, —(CH₂)_{z}—CN where z is 0-4, halo, —OH, —O—Rₐ, —OR_{b}, —CO—R_{c}, —O—CO—R_{c}, —CO—OR_{c}, —O—CO—OR_{c}, —O—CO—O—CO—R_{c}, —O—OR_{c}, keto (═O), thioketo (═S), —SO₂—R_{c}, —SO—R_{c}, —NR_{d}Rₑ, —CO—NR_{d}Rₑ, —O—CO—NR_{d}Rₑ, —NR_{c}—CO—NR_{d}Rₑ, —NR_{c}—CO—Rₑ, —NR_{c}—CO₂—ORₑ, —CO—NR_{c}—CO—R_{d}, —O—SO₂—R_{c}, —O—SO—R_{c}, —O—S—R_{c}, —S—CO—R_{c}, —SO—CO—OR_{c}, —SO₂—CO—OR_{c}, —O—SO₃H, —NR_{c}—SR_{d}, —NR_{c}—SO—R_{d}, —NR_{c}—SO₂—R_{d}, —CO—SR_{c}, —CO—SO—R_{c}, —CO—SO₂—R_{c}, —CS—R_{c}, —CSO—R_{c}, —CSO₂—R_{c}, —NR_{c}—CS—R_{d}, —O—CS—R_{c}, —O—CSO—R_{c}, —O—CSO₂—R_{c}, —SO₂—NR_{d}Rₑ, —SO—NR_{d}Rₑ, —S—NR_{d}Rₑ, —NR_{d}—CSO₂—R_{d}, —NR_{c}—CSO—R_{d}, —NR_{c}—CS—R_{d}, —SH, —S—R_{b}, and —PO₂—OR_{c}, where Rₐ is selected from the group consisting of alkyl, heteroalkyl, alkenyl, and alkynyl; R_{b} is selected from the group consisting of alkyl, heteroalkyl, haloalkyl, alkenyl, alkynyl, halo, —CO—R_{c}, —CO—OR_{c}, —O—CO—O—R_{c}, —O—CO—R_{c}, —NR_{c}—CO—R_{d}, —CO—NR_{d}Rₑ, —OH, aryl, heteroaryl, heterocycloalkyl, and cycloalkyl; Rc, Rd and Re are each independently selected from the group consisting of hydro, halo, alkyl, heteroalkyl, haloalkyl, alkenyl, alkynyl, —COR_{f}, —COOR_{f}, —O—CO—O—R_{f}, —O—CO—R_{f}, —OH, aryl, heteroaryl, cycloalkyl, and heterocycloalkyl, or R_{d} and Rₑ cyclize to form a heteroaryl or heterocycloalkyl group; and R_{f} is selected from the group consisting of hydro, alkyl, and heteroalkyl; and where any of the alkyl, heteroalkyl, alkenyl, aryl, cycloalkyl, heterocycloalkyl, or heteroaryl moieties present in the above substituents may be further substituted with one or more additional substituents independently selected from the group consisting of —NO₂, —NH₂, —CN, —(CH₂)_{z}—CN where z is 0-4, halo, haloalkyl, haloaryl, —OH, keto, —N(R_{c})OR_{d}, —NR_{d}Rₑ, —CO—NR_{d}Rₑ, —CO—OR_{c}, —CO—R_{c}, —NR_{c}—CO—NR_{d}Rₑ, —C—CO—OR_{c}, —NR_{c}—CO—R_{d}, —O—CO—O—R_{c}, —O—CO—NR_{d}Rₑ, —SH, —O—R_{b}, —O—Rₐ—O—R_{b}, —S—R_{b}, unsubstituted alkyl, unsubstituted aryl, unsubstituted cycloalkyl, unsubstituted heterocycloalkyl, and unsubstituted heteroaryl, where Rₐ, R_{b}, R_{c}, R_{d}, and Rₑ are as defined above;
R² and R⁵ are each independently hydro, halo, C₁₋₂ alkyl, —OCH₃, —OH, —NH₂, —NHCH₃, —N(CH₃)₂, —NO₂, —SH, —SCH₃, —S(O)CH₃, —SO₂CH₃, —P(CH₃)₂, or —PO₃H₂; and
R³ and R⁴ are each independently hydro, halo, methoxyl, or C₁₋₂ alkyl.

In the embodiment R¹ is a carboxamide, heterocycloalkyl or sulfonamide group unsubstituted or substituted as described above.

In another embodiment, R² and R⁵ are hydro.

In another embodiment, R³ and R⁴ are both halo positioned ortho relative to the point of attachment to the carbonyl. More preferably, R³ and R⁴ are fluoro.

In another preferred embodiment, the invention is directed to compounds represented by the Formula (II): 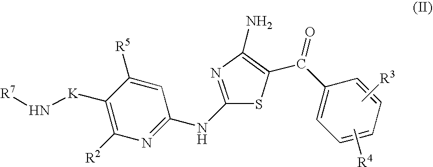
wherein
K is —C(O)— or —SO₂—;
R², R⁵, R³ and R⁴ are as defined in Formula (I) above; and
R⁷ is a C₁-₈ alkyl, C₁₋₈ alkylamino, aryl, C₁₋₈ alkyl-aryl, heteroaryl, heterocycloalkyl, C₁-₈-alkyl-heteroaryl, or C₁-₈ alkyl-heterocycloalkyl, unsubstituted or substituted with C₁-₈ alkyl, halo, methoxyl, aryl, or C₁₋₈ alkyl-aryl.

In yet another preferred embodiment, the compound is selected from the group consisting of:

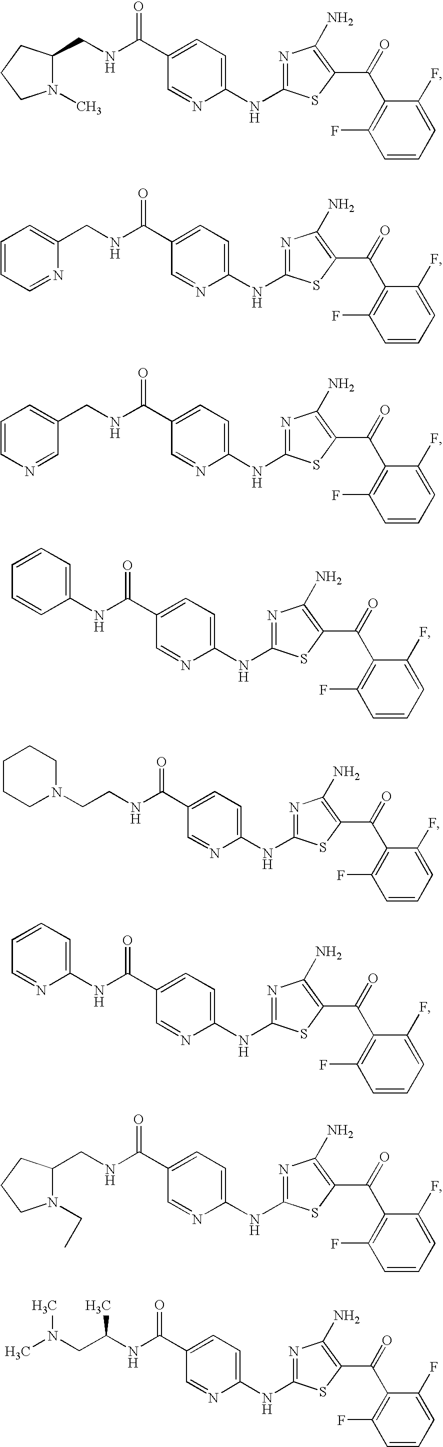

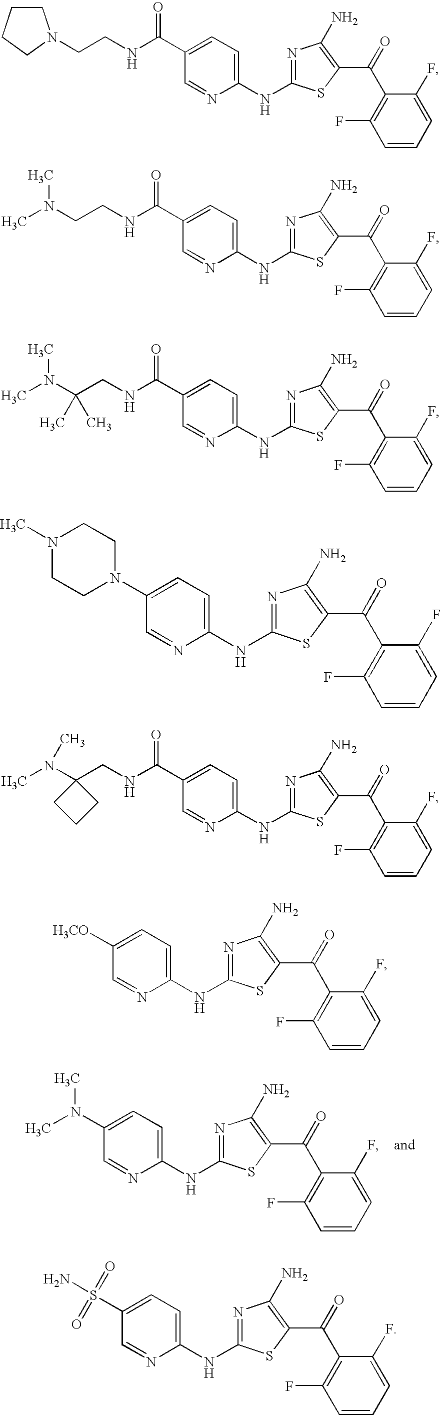

The invention is also directed to pharmaceutically acceptable prodrugs, multimeric forms, active metabolites, and pharmaceutically acceptable salts of compounds of Formulas (I) and (II) (such compounds, prodrugs, multimeric forms, metabolites and salts are collectively referred to as “agents”). Advantageous methods of making the compounds of the Formulas (I) and (II) are also described herein.

The invention also relates to pharmaceutical compositions comprising one or more compound(s) of Formula (I) and (II) or a pharmaceutically acceptable prodrug, multimeric form, active metabolite, or pharmaceutically acceptable salt thereof, in addition to a pharmaceutically acceptable carrier, diluent or vehicle. Preferably, the pharmaceutical compositions comprises an amount of an agent effective to modulate cellular proliferation and a pharmaceutically acceptable carrier. Alternately, the pharmaceutical composition comprises an amount of an agent effective to inhibit a protein kinase. In this alternate embodiment, the protein kinase is preferably a CDK2, CDK2/cyclin complex, CDK4, CDK4/cyclin complex.

The invention is also directed to methods of treating cellular proliferative diseases and other conditions associated with protein kinases, said methods comprising administering a therapeutically effective amount of at least one compound of Formulas (I) or (II) or a pharmaceutically acceptable prodrug, multimeric form, active metabolite, or pharmaceutically acceptable salt thereof to a subject in need of such treatment. In particular, the invention is directed to methods of treating conditions associated with a CDK2, CDK2/cyclin complex, CDK4, CDK4/cyclin complex. The inventive agents may be used to inhibit the development of certain cellular proliferative diseases, such as, for example, invasive cancer, tumor angiogenesis and metastasis. In particular, the inventive agents may be used for methods of treating familial melanoma, gliomas, leukemias, sarcomas, and pancreatic, non-small cell lung, and head and neck carcinomas.

Additional aspects, features, embodiments and advantages of the present invention will be apparent from the description that follows, or may be learned from practicing or using the present invention.

### DETAILED DESCRIPTION OF THE INVENTION

The terms “comprising” and “including” are used herein in their open, non-limiting sense.

As used herein, “cellular proliferative diseases” include diseases or disorders associated with uncontrolled or abnormal cellular proliferation. Such diseases and disorders include, but are not limited to, the following:
a variety of cancers, including, but not limited to, carcinoma, hematopoietic tumors of lymphoid lineage, hematopoietic tumors of myeloid lineage, tumors of mesenchymal origin, tumors of the central and peripheral nervous system and other tumors including melanoma, seminoma, and Kaposi's sarcoma and the like.
a disease process which features abnormal cellular proliferation, e.g., benign prostatic hyperplasia, familial adenomatosis polyposis, neuro-fibromatosis, atherosclerosis, pulmonary fibrosis, arthritis, psoriasis, glomerulonephritis, restenosis following angioplasty or vascular surgery, hypertrophic scar formation, inflammatory bowel disease, transplantation rejection, endotoxic shock, and fungal infections.
defective apoptosis-associated conditions, such as cancers (including, but not limited to, those types mentioned herein above), autoimmune diseases (including, but not limited to, systemic lupus erythematosus, rheumatoid arthritis, psoriasis, autoimmune mediated glomerulonephritis, inflammatory bowel disease and autoimmune diabetes mellitus), neurodegenerative disorders (including, but not limited to, Alzheimer's disease, amyotrophic lateral sclerosis, retinitis pigmentosa, Parkinson's disease, AIDS-related dementia, spinal muscular atrophy and cerebellar degeneration), myelodysplastic syndromes, aplastic anemia, ischemic injury associated with myocardial infarctions, stroke and reperfusion injury, arrhythmia, atherosclerosis, toxin-induced or alcohol related liver diseases, hematological diseases (including, but not limited to, chronic anemia and aplastic anemia), degenerative diseases of the musculoskeletal system (including, but not limited to, osteoporosis and arthritis), aspirin-sensitive rhinosinusitis, cystic fibrosis, multiple sclerosis, kidney diseases and cancer pain.

As used herein, “protein kinases” include, but are not limited to, cyclin-dependent kinases (CDKs), such as, for example, CDK2, CDK4, CDK5, CDK6 and/or cyclin complexes thereof, vascular endothelial growth factor receptor kinase (VEGF), fibroblast growth factor (FGF), lymphocyte control kinase (LCK), protein kinase C (PKC), her2, rafl, MEK1, mitogen activated protein (MAP) kinase, endothelial growth factor (EGF) receptor kinase, platelet-derived growth factor (PDGF) receptor kinase, insulin growth factor (IGF) receptor kinase, phosphatidyl inositol-3 (PI3) kinase, weel kinase, Src, and/or Abl.

As used herein, “diseases associated with protein kinases” include, but are not limited to, viral infections such as, for example, HIV, human papilloma virus, herpesvirus, Epstein-Barr virus, adenovirus, Sindbis virus, poxvirus and the like, and neurodegenerative diseases, such as, for example, Alzheimer's disease.

The term “alkyl” refers to a straight- or branched-chain alkyl group having from 1 to 12 carbon atoms in the chain. Exemplary alkyl groups include methyl (Me, which also may be structurally depicted by /), ethyl (Et), n-propyl, isopropyl, butyl, isobutyl, sec-butyl, tert-butyl (tBu), pentyl, isopentyl, tert-pentyl, hexyl, isohexyl, and the like. The term “C₁₋₈ alkyl” refers to a straight- or branched-chain alkyl group having one to eight carbon atoms in the chain. Similarly, the term “C₁₋₂ alkyl” refers to a straight- or branched-chain alkyl group having one or two carbon atoms in the chain.

The term “heteroalkyl” refers to a straight- or branched-chain alkyl group having from 2 to 12 atoms in the chain, one or more of which is a heteroatom selected from S, O, and N. Exemplary heteroalkyls include alkoxyls, alcohols, esters, alkyl ethers, secondary and tertiary alkyl amines, alkyl sulfides, and the like.

The term “alkenyl” refers to a straight- or branched-chain alkenyl group having from 2 to 12 carbon atoms in the chain. Illustrative alkenyl groups include prop-2-enyl, but-2-enyl, but-3-enyl, 2-methylprop-2-enyl, hex-2-enyl, ethenyl, pentenyl, and the like.

The term “alkynyl” refers to a straight- or branched-chain alkynyl group having from 2 to 12 carbon atoms in the chain. Illustrative alkynyl groups include prop-2-ynyl, but-2-ynyl, but-3-ynyl, 2-methylbut-2-ynyl, hex-2-ynyl, ethynyl, propynyl, pentynyl and the like.

The term “aryl” (Ar) refers to a monocyclic, or fused polycyclic, aromatic carbocycle (ring structure having ring atoms that are all carbon) having from 3 to 12 ring atoms per ring. Illustrative examples of aryl groups include the following moieties:

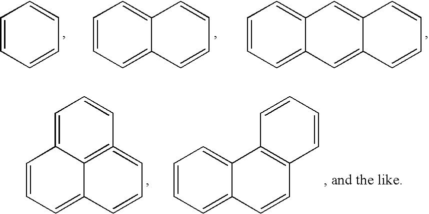

The term a “C₁-C₈ alkylamino” refers to an amino group attached to one or more alkyl groups, each alkyl group being a straight- or branched-chain alkyl having 1 to 8 carbon atoms in the chain. Exemplary alkylamino groups include —N(CH₃)₂, —NHCH₃, —NHCH₂CH₃.

The term “heteroaryl” (heteroAr) refers to a monocyclic, or fused polycyclic, aromatic heterocycle (ring structure having ring atoms selected from carbon atoms as well as nitrogen, oxygen, and sulfur heteroatoms) having from 3 to 12 ring atoms per ring. Illustrative examples of heteroaryl groups include moieties having 4 to 7 ring atoms per ring, such as the following moieties:

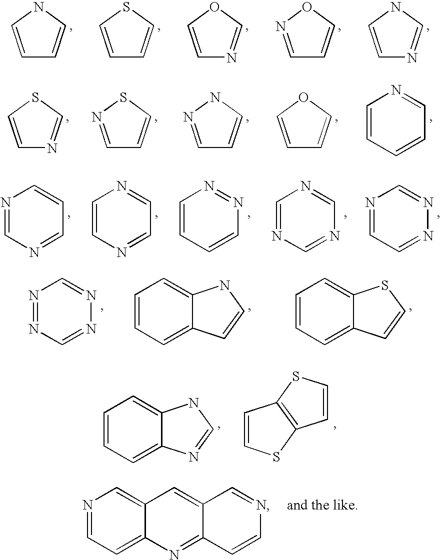

The term “cycloalkyl” refers to a saturated or partially saturated, monocyclic or fused or spiro polycyclic, carbocycle having from 3 to 12 ring atoms per ring. Illustrative examples of cycloalkyl groups include cycloalkyl groups having 4 to 8 ring atoms per ring, such as the following moieties:

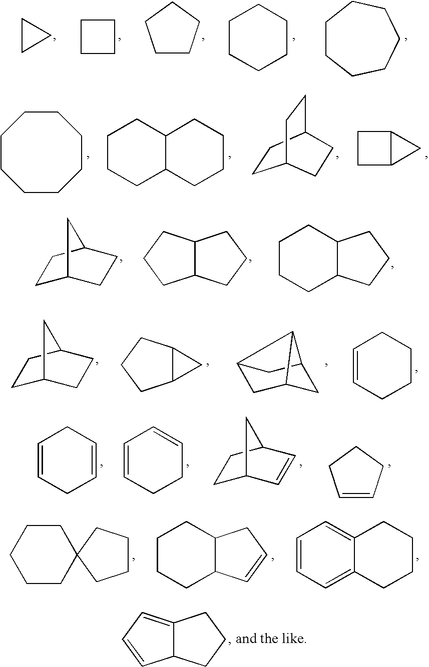

A “heterocycloalkyl” refers to a monocyclic, or fused or spiro polycyclic, ring structure that is saturated or partially saturated and has from 3 to 12 ring atoms per ring selected from C atoms and N, O, and S heteroatoms. Illustrative examples of heterocycloalkyl groups include heterocycloalkyl groups having 4 to 8 ring atoms per ring, such as the following:

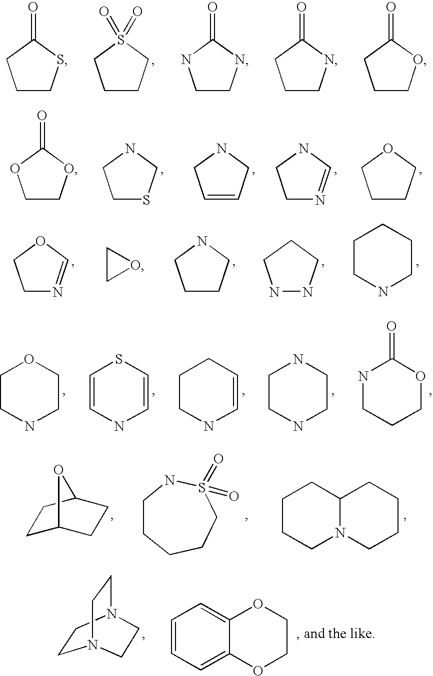

The term “halogen” represents chlorine, fluorine, bromine or iodine. The term “halo” represents chloro, fluoro, bromo or iodo. The term “haloalkyl” refers to an alkyl group as defined above containing one or more chloro, fluoro, bromo or iodo atoms (or combinations thereof. The term “haloaryl” refers to an aryl group as defined above containing one or more chloro, fluoro, bromo or iodo atoms (or combinations thereof. The term “halocycloalkyl” refers to a cycloalkyl group as defined above containing one or more chloro, fluoro, bromo or iodo atoms (or combinations thereof. The term “haloheterocycloalkyl” refers to a heterocycloalkyl group as defined above containing one or more chloro, fluoro, bromo or iodo atoms (or combinations thereof.

An “alkoxy group” is intended to mean the radical —ORₖ, where Rₖ is an alkyl group. Exemplary alkoxy groups include methoxy, ethoxy, and propoxy. “Lower alkoxy” refers to alkoxy groups wherein the alkyl portion has 1 to 4 carbon atoms.

The term “carboxamide” refers to the radical —C(O)N(R′)(R″) where R′ and R″ are each independently selected from hydrogen, —OH and alkyl, alkenyl, alkynyl, alkoxy, cycloalkyl, heterocycloalkyl, heteroaryl, aryl groups as defined above; or R′ and R″ cyclize together with the nitrogen to form a heterocycloalkyl or heteroaryl as defined above.

The term “sulfonamide” refers to the radical —S(O)₂N(R′)(R″) where R′ and R″ are each independently selected from hydrogen, —OH and alkyl, alkenyl, alkynyl, alkoxy, cycloalkyl, heterocycloalkyl, heteroaryl, aryl groups as defined above; or R′ and R″ cyclize together with the nitrogen to form a heterocycloalkyl or heteroaryl as defined above.

In accordance with a convention used in the art, 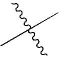
is used in structural formulae herein to depict the bond that is the point of attachment of the moiety or substituent to the core or backbone structure. Moreover, 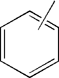
is used in structural formulae herein to depict that the point of attachment of the moiety or substituent to the core of the backbone aryl structure is unspecified. Where chiral carbons are included in chemical structures, unless a particular orientation is depicted, both stereoisomenc forms are intended to be encompassed. Further, the specific inhibitors of the present invention may exist as single stereoisomers, racemates, and/or mixtures of enantiomers and/or diastereomers. All such single stereoisomers, racemates, and mixtures thereof are intended to be within the broad scope of the present invention. The chemical formulae referred to herein may exhibit the phenomenon of tautomerism. Although the structural formulae depict one of the possible tautomeric forms, it should be understood that the invention nonetheless encompasses all tautomeric forms.

The term “substituted” means that the specified group or moiety bears one or more substituents. The term “unsubstituted” means that the specified group bears no substituents. The term “optionally substituted” means that the specified group is unsubstituted or substituted by one or more substituents. The term “substituent” or “suitable substituent” is intended to mean any suitable substituent that may be recognized or selected, such as through routine testing, by those skilled in the art. Unless expressly indicated otherwise, illustrative examples of suitable substituents include alkyl, heteroalkyl, haloalkyl, haloaryl, halocycloalkyl, haloheterocycloalkyl, aryl, cycloalkyl, heterocycloalkyl, heteroaryl, —NO₂, —NH₂, —N—(R_{c})OR_{d}, —CN, —(CH₂)_{z}—CN where z is 0-4, halo, —OH, —O—Rₐ, —OR_{b}, —CO—R_{c}, —O—CO—R_{c}, —CO—OR_{c}, —O—CO—OR_{c}, —O—CO—O—CO—R_{c}, —O—OR_{c}, keto (═O), thioketo (═S), —SO₂—R_{c}, —SO—R_{c}, —NR_{d}Rₑ, —CO—NR_{d}Rₑ, —O—CO—NR_{d}Rₑ, —NR_{c}—CO—NR_{d}Rₑ, —NR_{c}—CO—Rₑ, —NR_{c}—CO₂—ORₑ, —CO—NR_{c}—CO—R_{d}, —O—SO₂—R_{c}, —O—SO—R_{c}, —O—S—R_{c}, —S—CO—R_{c}, —SO—CO—OR_{c}, —SO₂—CO—OR_{c}, —O—SO₃H, —NR_{c}—SR_{d}, —NR_{c}—SO—R_{d}, —NR_{c}—SO₂—R_{d}, —CO—SR_{c}, —CO—SO—R_{c}, —CO—SO₂—R_{c}, —CS—R_{c}, —CSO—R_{c}, —CSO₂—R_{c}, —NR_{c}—CS—R_{d}, —O—CS—R_{c}, —O—CSO—R_{c}, —O—CSO₂—R_{c}, —SO₂—NR_{d}Rₑ, —SO—NR_{d}Rₑ, —S—NR_{d}Rₑ, —NR_{d}—CSO₂—R_{d}, —NR_{c}—CSO—R_{d}, —NR_{c}—CS—R_{d}, —SH, —S—R_{b}, and —PO₂—OR_{c}, where Rₐ is selected from the group consisting of alkyl, heteroalkyl, alkenyl, and alkynyl; R_{b} is selected from the group consisting of alkyl, heteroalkyl, haloalkyl, alkenyl, alkynyl, halo, —CO—R_{c}, —CO—OR_{c}, —O—CO—O—R_{c}, —O—CO—R_{c}, —NR_{c}—CO—R_{d}, —CO—NR_{d}Rₑ, —OH, aryl, heteroaryl, heterocycloalkyl, and cycloalkyl; R_{c}, R_{d} and Rₑ are each independently selected from the group consisting of hydro, halo, alkyl, heteroalkyl, haloalkyl, alkenyl, alkynyl, —COR_{f}, —COOR_{f}, —O—CO—O—R_{f}, —O—CO—R_{f}, —OH, aryl, heteroaryl, cycloalkyl, and heterocycloalkyl, or R_{d} and Rₑ cyclize to form a heteroaryl or heterocycloalkyl group; and R_{f} is selected from the group consisting of hydro, alkyl, and heteroalkyl; and where any of the alkyl, heteroalkyl, alkenyl, aryl, cycloalkyl, heterocycloalkyl, or heteroaryl moieties present in the above substituents may be further substituted with one or more additional substituents independently selected from the group consisting of —NO₂, —NH₂, —CN, —(CH₂)_{z}—CN where z is 0-4, halo, haloalkyl, haloaryl, —OH, keto (═O), —N—(R_{c})OR_{d}, —NR_{d}Rₑ, —CO—NR_{d}Rₑ, —CO—OR_{c}, —CO—R_{c}, —NR_{c}—CO—NR_{d}Rₑ, —C—CO—OR_{c}, —NR_{c}—CO—R_{d}, —O—CO—O—R_{c}, —O—CO—NR_{d}Rₑ, —SH, —O—R_{b}, —O—RₐO—R_{b}, —S—R_{b}, unsubstituted alkyl, unsubstituted aryl, unsubstituted cycloalkyl, unsubstituted heterocycloalkyl, and unsubstituted heteroaryl, where Rₐ, R_{b}, R_{c}, R_{d}, and Rₑ are as defined above.

Preferred compounds of the invention include the following compounds, as well as any other compound(s) described in the Examples below:

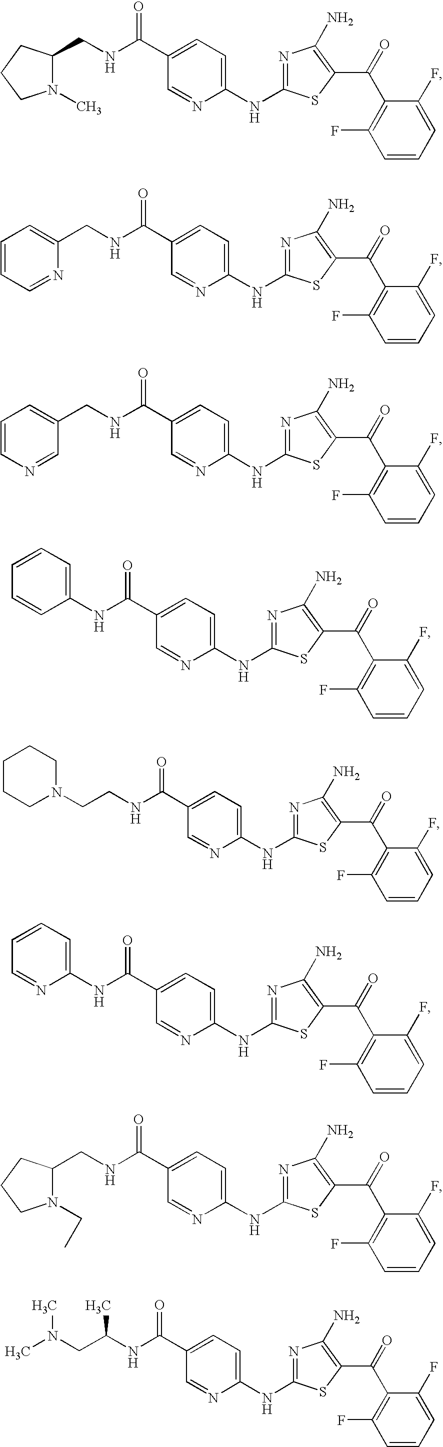

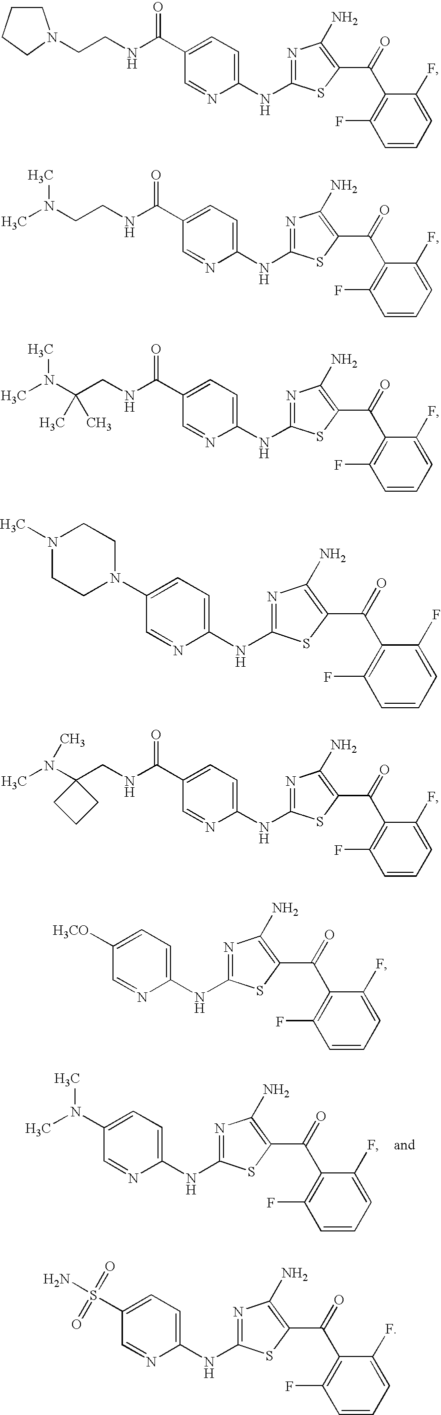

Some of the inventive agents may exist in various stereoisomeric or tautomeric forms. The present invention encompasses all such cell proliferation-inhibiting compounds, including active compounds in the form of single pure enantiomers (i.e., essentially free of other stereoisomers), racemates, mixtures of enantiomers and/or diastereomers, and/or tautomers. Preferably, the inventive compounds that are optically active are used in optically pure form.

As generally understood by those skilled in the art, an optically pure compound having one chiral center (i.e., one asymmetric carbon atom) is one that consists essentially of one of the two possible enantiomers (i.e., is enantiomerically pure), and an optically pure compound having more than one chiral center is one that is both diastereomerically pure and enantiomerically pure.

Preferably, the compounds of the present invention are used in a form that is at least 90% optically pure, that is, a form that contains at least 90% of a single isomer (80% enantiomeric excess (“e.e.”) or diastereomeric excess (“d.e.”)), more preferably at least 95% (90% e.e. or d.e.), even more preferably at least 97.5% (95% e.e. or d.e.), and most preferably at least 99% (98% e.e. or d.e.).

Additionally, the formulae are intended to cover solvated as well as unsolvated forms of the identified structures. For example, Formula I includes compounds of the indicated structure in both hydrated and non-hydrated forms. Other examples of solvates include the structures in combination with isopropanol, ethanol, methanol, DMSO, ethyl acetate, acetic acid, or ethanolamine.

In addition to compounds of Formula I, the invention includes pharmaceutically acceptable prodrugs, multimeric forms, pharmaceutically active metabolites, and pharmaceutically acceptable salts of such compounds and metabolites.

The term “pharmaceutically acceptable” means pharmacologically acceptable and substantially non-toxic to the subject being administered the cell-cycle control agent.

A “prodrug” is a compound that may be converted under physiological conditions or by solvolysis to the specified compound or to a pharmaceutically acceptable salt of such compound. An “active metabolite” is a pharmacologically active product produced through metabolism in the body of a specified compound or salt thereof. Prodrugs and active metabolites of a compound may be identified using routine techniques known in the art. See, e.g., Bertolini et al., J. Med. Chem., (1997) 40:2011-2016; Shan et al., J. Pharm. Sci., 86 (7):765-767; Bagshawe, Drug Dev. Res., (1995) 34:220-230; Bodor, Advances in Drug Res., (1984) 13:224-331; Bundgaard, Design of Prodrugs (Elsevier Press 1985); Larsen, Design and Application of Prodrugs, Drug Design and Development (Krogsgaard-Larsen et al. eds., Harwood Academic Publishers, 1991); Dear et al., J. Chromatogr. B, (2000) 748:281-293; Spraul et al., J. Pharmaceutical & Biomedical Analysis, (1992) 10 (8):601-605; and Prox et al., Xenobiol, (1992) 3 (2):103-112.

A “solvate” is intended to mean a pharmaceutically acceptable solvate form of a specified compound that retains the biological effectiveness of such compound. Examples of solvates include compounds of the invention in combination with water, isopropanol, ethanol, methanol, DMSO, ethyl acetate, acetic acid, or ethanolamine.

The affinity of the compounds of the invention for a receptor may be enhanced by providing multiple copies of the ligand in close proximity, preferably using a scaffolding provided by a carrier moiety. Such multivalent or multimers of active forms of the compounds of the Formula I or II are referred to herein as “multimeric forms”. Multimers of various dimensions (i.e., bearing varying numbers of copies of an active compound) may be tested to arrive at a multimer of optimum size with respect to receptor binding. Provision of such multivalent forms of active receptor-binding compounds with optimal spacing between the receptor-binding moieties may enhance receptor binding (see, for example, Lee, R. T.; et al., Biochem., 1984, 23, 4255-4261). The artisan may control the multivalency and spacing by selection of a suitable carrier moiety or linker units. Useful moieties include molecular supports containing a multiplicity of functional groups that can be reacted with functional groups associated with the active compounds of the invention. A variety of carrier moieties may be used to build highly active multimers, including proteins such as BSA (bovine serum albumin) or HAS, peptides such as pentapeptides, decapeptides, pentadecapeptides, and the like, as well as non-biological compounds selected for their beneficial effects on absorbability, transport, and persistence within the target organism. Functional groups on the carrier moiety, such as amino, sulfhydryl, hydroxyl, and alkylamino groups, may be selected to obtain stable linkages to the compounds of the invention, optimal spacing between the immobilized compounds, and optimal biological properties.

A “pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, phosphoric acid and the like, or with an organic acid, such as acetic acid, phenylacetic acid, propionic acid, stearic acid, lactic acid, ascorbic acid, maleic acid, hydroxymaleic acid, isethionic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid, 2-acetoxybenzoic acid or cinnamic acid, a sulfonic acid, such as p-toluenesulfonic acid, methansulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine and piperazine, and inorganic salts derived from sodium, calcium, potassium, magnesium, manganese, iron, copper, zinc, aluminum and lithium.

Pharmaceutical compositions according to the invention may, alternatively or in addition to a compound of Formulas I and II, comprise as an active ingredient pharmaceutically acceptable prodrugs, multimeric forms, pharmaceutically active metabolites, and pharmaceutically acceptable salts of such compounds and metabolites. Such compounds, prodrugs, multimers, salts, and metabolites are sometimes referred to herein collectively as “active agents” or “agents.”

In the case of agents that are solids, it is understood by those skilled in the art that the inventive compounds and salts may exist in different crystal or polymorphic forms, all of which are intended to be within the scope of the present invention and specified formulas.

Therapeutically effective amounts of the active agents of the invention may be used to treat and/or prevent diseases mediated by modulation or regulation of various kinases, for example protein kinases or to treat and/or prevent cellular proliferative diseases. An “effective amount” is intended to mean that amount of an agent that significantly inhibits proliferation and/or prevents de-differentiation of a eukaryotic cell, e.g., a mammalian, insect, plant or fungal cell, and is effective for the indicated utility, e.g., specific therapeutic treatment.

The amount of a given agent that will correspond to such an amount will vary depending upon factors such as the particular compound, disease condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. “Treating” is intended to mean at least the mitigation of a disease condition in a subject such as mammal (e.g., human), that is affected, at least in part, by the activity of one or more kinases, for example protein kinases such as tyrosine kinases, and includes: preventing the disease condition from occurring in a mammal, particularly when the mammal is found to be predisposed to having the disease condition but has not yet been diagnosed as having it; modulating and/or inhibiting the disease condition; and/or alleviating the disease condition.

Agents that potently regulate, modulate, or inhibit cell proliferation are preferred. For certain mechanisms, inhibition of the protein kinase activity associated with CDK complexes, among others, and those which inhibit angiogenesis and/or inflammation are preferred. The present invention is further directed to methods of modulating or inhibiting protein kinase activity, for example in mammalian tissue, by administering an inventive agent. The activity of agents as anti-proliferatives is easily measured by known methods, for example by using whole cell cultures in a colorimetric based assay with 3-(4,5-dimethylthiazol-2-yl)-2,5-diphenyl-tetrazolium bromide (MTT) assay (Mosmann, T. J. Immunol Methods 1983, 65, 55-63.). The activity of the inventive agents as modulators of protein kinase activity, such as the activity of kinases, may be measured by any of the methods available to those skilled in the art, including in vivo and/or in vitro assays. Examples of suitable assays for activity measurements include those described in WIPO International Publication No. WO 99/21845; Parast, C.; et al.; Biochemistry, 1998, 37, 16788-16801; Connell-Crowley, L.; Harper, W.; in Cell Cycle: Materials and Methods, Pagano, M.; ed. Springer, Berlin, Germany, 1995, 157-168.; WIPO International Publication No. WO 97/34876; and WIPO International Publication No. WO 96/14843. These properties may be assessed, for example, by using one or more of the biological testing procedures set out in the examples below.

The active agents of the invention may be formulated into pharmaceutical compositions as described below. Pharmaceutical compositions of this invention comprise an effective modulating, regulating, or inhibiting amount of at least one inventive agent and an inert, pharmaceutically acceptable carrier or diluent. In one embodiment of the pharmaceutical compositions, efficacious levels of the inventive agents are provided so as to provide therapeutic benefits involving anti-proliferative ability. By “efficacious levels” is meant levels in which proliferation is inhibited, or controlled. These compositions are prepared in unit-dosage form appropriate for the mode of administration, e.g., parenteral or oral administration.

An inventive agent can be administered in conventional dosage form prepared by combining a therapeutically effective amount of an agent (e.g., a compound of Formula I) as an active ingredient with appropriate pharmaceutical carriers or diluents according to conventional procedures. These procedures may involve mixing, granulating and compressing or dissolving the ingredients as appropriate to the desired preparation.

The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of an inventive agent can be dissolved in an aqueous solution of an organic or inorganic acid, such as 0.3M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, glycerin and the like in concentrations ranging from 0-60% of the total volume. In an exemplary embodiment, a compound of Formula I is dissolved in DMSO and diluted with water. The composition may also be in the form of a solution of a salt form of the active ingredient in an appropriate aqueous vehicle such as water or isotonic saline or dextrose solution.

It will be appreciated that the actual dosages of the agents used in the compositions of this invention will vary according to the particular complex being used, the particular composition formulated, the mode of administration and the particular site, host and disease being treated. Optimal dosages for a given set of conditions can be ascertained by those skilled in the art using conventional dosage-determination tests in view of the experimental data for an agent. For oral administration, an exemplary daily dose generally employed is from about 0.001 to about 1000 mg/kg of body weight, with courses of treatment repeated at appropriate intervals. Administration of prodrugs is typically dosed at weight levels which are chemically equivalent to the weight levels of the fully active form.

The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations which can be used pharmaceutically.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active agents in water-soluble form. Additionally, suspensions of the agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

For administration to the eye, the active agent is delivered in a pharmaceutically acceptable ophthalmic vehicle such that the compound is maintained in contact with the ocular surface for a sufficient time period to allow the compound to penetrate the corneal and internal regions of the eye, including, for example, the anterior chamber, posterior chamber, vitreous body, aqueous humor, vitreous humor, cornea, iris/ciliary, lens, choroid/retina and sclera. The pharmaceutically acceptable ophthalmic vehicle may be an ointment, vegetable oil, or an encapsulating material. A compound of the invention may also be injected directly into the vitreous and aqueous humor.

Alternatively, the active agents may be in powder form for constitution with a suitable vehicle, e.g., sterile pyrogen-free water, before use. The compounds may also be formulated in rectal compositions such as suppositories or retention enemas, e.g, containing conventional suppository bases such as cocoa butter or other glycerides.

In addition to the formulations described above, the active agents can also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

An examplary pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the compounds of the invention may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other protonic solvents than are the corresponding free-base forms.

The active agents of the invention may be useful in combination with known anti-cancer treatments such as, but not limited to, DNA interactive agents such as cisplatin or doxorubicin; topoisomerase II inhibitors such as etoposide, topoisomerase I inhibitors such as CPT-11 or topotecan; tubulin interacting agents such as paclitaxel, docetaxel or the epothilones; hormonal agents such as tamoxifen; thymidilate synthase inhibitors such as 5-fluorouracil; and anti-metalbolites such as methotrexate. They may be administered together or sequentially, and when administered sequentially, the inventive agents may be administered either prior to or after administration of the known anticancer or cytotoxic agent.

The inventive agents may be prepared using the reaction routes and synthesis schemes as described below, employing the general techniques known in the art using starting materials that are readily available. The preparation of preferred compounds of the present invention is described in detail in the following examples, but the artisan will recognize that the chemical reactions described may be readily adapted to prepare a number of other agents of the invention. For example, the synthesis of non-exemplified compounds according to the invention may be successfully performed by modifications apparent to those skilled in the art, e.g., by appropriately protecting interfering groups, by changing to other suitable reagents known in the art, or by making routine modifications of reaction conditions. Alternatively, other reactions disclosed herein or generally known in the art will be recognized as having applicability for preparing other compounds of the invention.

### EXAMPLES

The present invention will be further illustrated in the following, non-limiting examples.

In the examples described below, unless otherwise indicated, all temperatures are set forth in degrees Celsius and all parts and percentages are by weight. Reagents were purchased from commercial suppliers, such as Aldrich Chemical Company or Lancaster Synthesis Ltd. and were used without further purification unless otherwise indicated. Tetrahydrofuran (THF) and N,N-dimethylformamide (DMF) were purchased from Aldrich in Sure Seal bottles and used as received. All solvents were purified using standard methods known to those skilled in the art, unless otherwise indicated.

The reactions set forth below were done generally under a positive pressure of argon at an ambient temperature (unless otherwise stated) in anhydrous solvents, and the reaction flasks were fitted with rubber septa for the introduction of substrates and reagents via syringe. Glassware was oven dried and/or heat dried. Analytical thin layer chromatography (TLC) was performed on glass-backed silica gel 60 F 254 plates from Analtech (0.25 mm), eluted with the appropriate solvent ratios (v/v), and are denoted where appropriate. The reactions were assayed by TLC, HPLC, or ¹H NMR, and terminated as judged by the consumption of starting material.

Visualization of the TLC plates was done with iodine vapor, ultraviolet illumination, 2% Ce(NH₄)₄(SO₄)₄ in 20% aqueous sulfuric acid, 2% ninhydrin in ethanol, or p-anisaldehyde spray reagent, and activated with heat where appropriate. Work-ups were typically done by doubling the reaction volume with the reaction solvent or extraction solvent and then washing with the indicated aqueous solutions using 25% by volume of the extraction volume unless otherwise indicated. Product solutions were dried over anhydrous Na₂SO₄ and/or MgSO₄ prior to filtration and evaporation of the solvents under reduced pressure on a rotary evaporator and noted as solvents removed in vacuo. When indicated, column chromatography refers to the flash column chromatography protocol of Still, W. C.; Kahn, M.; Mitra, A. J. Org. Chem., 1978, 43, 2923-2925 that employs Merck silica gel (47-61 μm) with a silica gel crude material ratio of about 20:1 to 50:1, unless otherwise stated. Certain example compounds were purified via preparative high-performance liquid chromatography (HPLC), and unless otherwise indicated, refers to a Gilson 321 or Dionex Summit system, equipped with a C18 reversed-phase preparative column (Metasil AQ 10 micron, 120A, 250×21.2 mm, MetaChem or Dionex Acclaim 120, respectively) and elution with a gradient of 0.1% trifluoroacetic acid (TFA)/5% acetonitrile/water to 0.1% TFA/5% water/acetonitrile over 20 min and flow rate of 20 mL/min. Hydrogenations were performed at ambient pressure unless otherwise indicated. All melting points (mp) are uncorrected.

¹H-NMR spectra were recorded on a Bruker or Varian instrument operating at 300 or 400 MHz and ¹³C-NMR spectra were recorded operating at 75 MHz. NMR spectra were obtained as CDCl₃ solutions (reported in ppm), using chloroform as the reference standard (7.27 ppm and 77.00 ppm) unless otherwise indicated. When peak multiplicities are reported, the following abbreviations are used: s (singlet), d (doublet), t (triplet), q (quartet), m (multiplet), bs (broad singlet), bm (broad multiplet), dd (doublet of doublets), ddd (doublet of doublet of doublets), dddd (doublet of doublet of doublet of doublets), dt (doublet of triplets). Coupling constants, when given, are reported in Hertz (Hz). Mass spectrometry was conducted with various techniques. Matrix-Assisted Laser Desorption/Ionization Fourier Transform Mass Spectrometry (MALDI FTMS), was performed on an IonSpec FTMS mass spectrometer. Samples are irradiated with a nitrogen laser (Laser Science Inc.) operated at 337 nm and the laser beam is attenuated by a variable attenuator and focused on the sample target. The ions are then differentiated according to their m/z using an ion cyclotron resonance mass analyzer. The electrospray ionization (ESI) mass spectrometry experiments were performed on an API 100 Perkin Elmer SCIEX single quadrupole mass spectrometer. Electrospray samples are typically introduced into the mass analyzer at a rate of 4.0 μl/minute. The positive and negative ions, generated by charged droplet evaporation, enter the analyzer through an interface plate and a 100 mm orifice, while the declustering potential is maintained between 50 and 200V to control the collisional energy of the ions entering the mass analyzer. The emitter voltage is typically maintained at 4000V. The liquid chromatography (LC) electrospray ionization (ESI) or atmospheric pressure chemical ionization (APCI) mass spectrometry experiments were performed on an Hewlett-Packard (HP) 1100 MSD single quadrupole mass spectrometer. Electrospray samples are typically introduced into the mass analyzer at a rate of 100 to 1000 μl/minute. The positive and negative ions, generated by charged droplet evaporation, enter the analyzer through a heated capillary plate, while the declustering potential is maintained between 100 and 300V to control the collisional energy of the ions entering the mass analyzer. The emitter voltage is typically maintained at 4000V.

General Methods of Preparation

As shown below in Scheme I, structural amides 1-3 described herein arose from straightforward amide formation via coupling of primary amines 1-2 with the pendant acids on thiazoles I-1. Typical coupling reagents and corresponding conditions were employed, such as N-(3-dimethylaminopropyl)-N′-ethylcarbodiimide hydrochloride (EDC), O-(7-azabenzotriazol-1-yl)-1,1,3,3-tetramethyluronium hexafluorophosphate (HATU), (benzotriazol-1-yloxy)tripyrrolidinophosphonium hexafluorophosphate (pyBOP), 2-chloro-4,6-dimethoxy-1,3,5-triazine (Kaminski, Z. J. Synthesis 1987, 917-20), or 4-(4,6-dimethoxy-1,3,5-triazin-2-yl)-4-methylmorpholinium chloride (Kunishima, M.; Kawachi, C.; Morita, J.; Terao, K.; Iwasaki, F.; Tani, S. Tetrahedron 1999, 55, 13159-13170), or many others that would be familiar to those skilled in the art. Obviously the use of the most efficient conditions would allow for parallel preparation, or combinatorial libraries of such amides I-3.

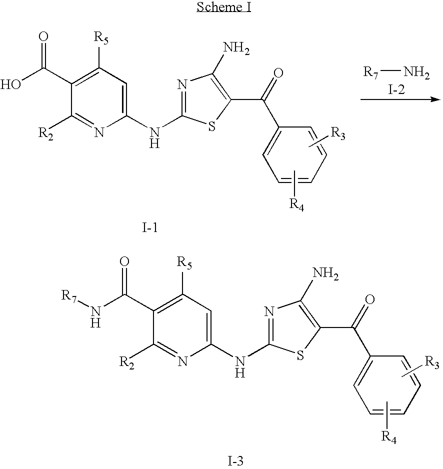

The requisite acids I-1 were produced from saponification of the corresponding esters II-1, typically ethyl esters. As shown in Scheme II, the convergence of three components culminates in the cyclization to the thiazole ring, as best exemplified in the literature by Gewald, K.; Blauschmidt, P.; Mayer, R. J. Prakt. Chem. 1967, 35, 97-104 and World Patent Application WO99/21845. The initial condensation of isothiocyanates II-2 and cyanamide (II-3) in the presence of a base, provides negatively charged isothiourea II-5, which is treated in the same reaction vessel with □-haloacetophenone II-4. The resultant S-alkyl-isothiourea II-6 is deprotonated in the basic medium to effect cyclization to the thiazole II-1. The bases employed can range from the simple alkoxides, as in the original Gewald protocol, to more hindered, non-nucleophilic bases such as potassium tert-butoxide or 1,8-diazabicyclo[5.4.0]-undec-7-ene (DBU) that are more appropriate for the functionalities present in many of the examples. The starting components for the thiazoles, n-haloacetophenone II-4 and its relatives, are available from multiple methods as described (in detail) in World Patent Application WO99/21845. The pyridyl-isothiocyanates II-2 were also prepared with methods culled from the prior citation, but explicitly detailed herein. The corresponding amino-pyridines from the literature underwent treatment with thiophosgene, under either alkaline or acidic conditions as warranted.

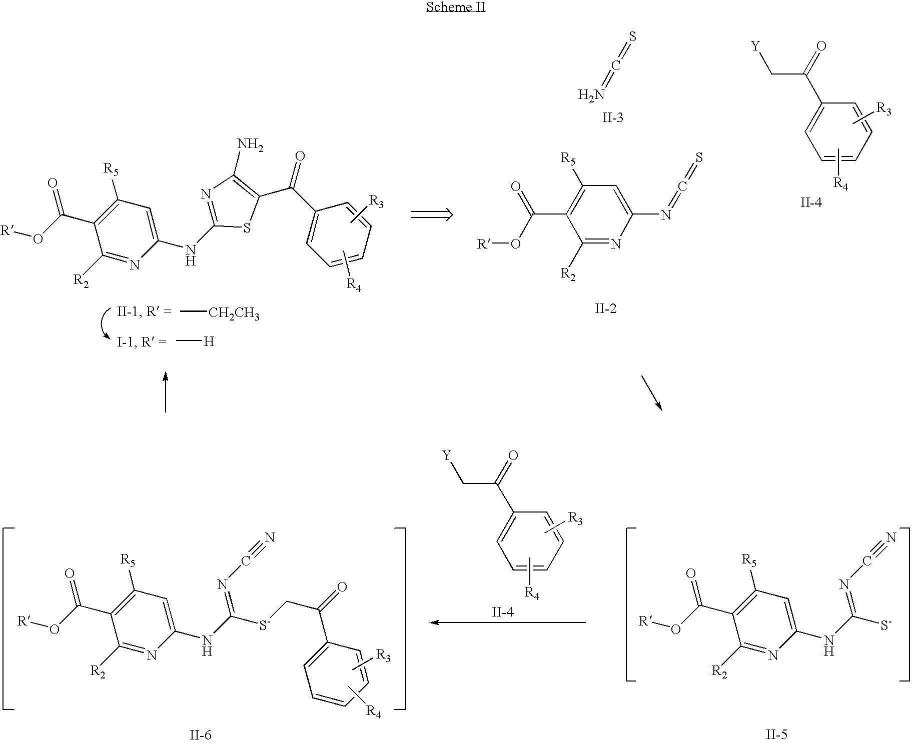

As shown in Scheme III below, the isothiocyanates II-2 for Scheme II were prepared upon conversion of corresponding amines with traditional methods—typically thiophosgene, with either alkaline or acidic conditions as warranted, and as described in the prior citation WO99/21845. Other methods can prepare isothiocyanates, such as treatment of amines with carbon disulfide in either acidic or alkaline media, or with hydrogen peroxide, see Li, G.; Tajima, H.; Ohtani, T. J. Org. Chem. 1997, 62, 4539-4540 and references therein. Specific examples are explicitly detailed herein.

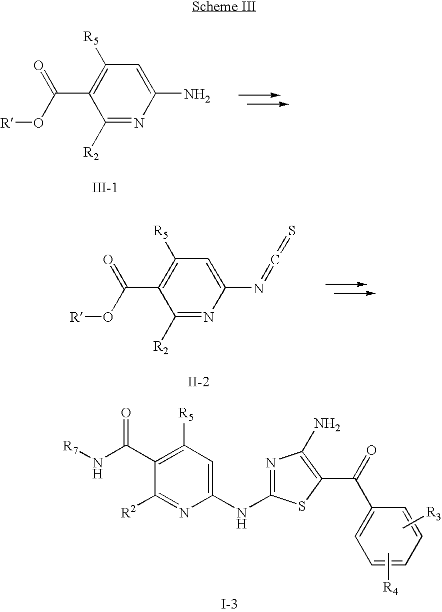

Certain carboxamides may require earlier introduction in the sequence, for example, as illustrated in Scheme IV. Anilide formation can be a challenge in the amide formation shown in Scheme I, so anilide-thiazole I-3AR could be prepared from the requisite anilide-isothiocyanate IV-1, which in turn evolves from the nitropyridyl-anilide IV-2. The anilide is formed in an initial condensation of anilines IV-4, particularly amino-heteroaryls such as aminopyridines, with the acid chlorides IV-3 from the literature and those described in detail below.

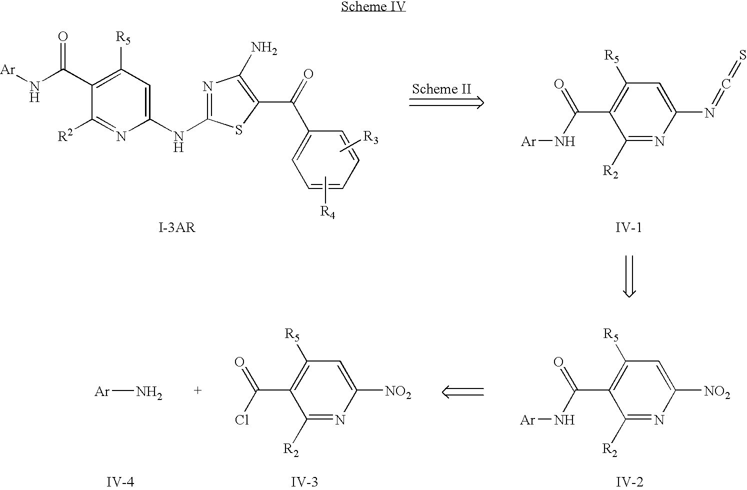

Other examples with various substitution patterns on the pyridine ring can be prepared as seen in the following Schemes V through VIII. For example, target I-3A can be made as depicted in Scheme V: amino-pyridines V-1 from the literature can be converted to corresponding isothiocyanates II-2, which in turn lead to targets 1-3 as described in Scheme III. A 2,3,6-trisubstituted pyridine III-1B is obtained from displacement of fluoride in III-1A by methoxide. The amine in III-1A arose from ammonia substituting difluoride V-1, which originated from capping the resultant anion of commercially available 2,6-difluoro-pyridine (V-2), upon exposure to an alkyllithium at low temperature. This sequence to III-1B was elegantly used in Coldwell, M. C.; Gadre, A.; Jerman, J.; King, F. D.; Nash, D. Bioorg. Med. Chem. Lett. 1995, 5, 39-42.

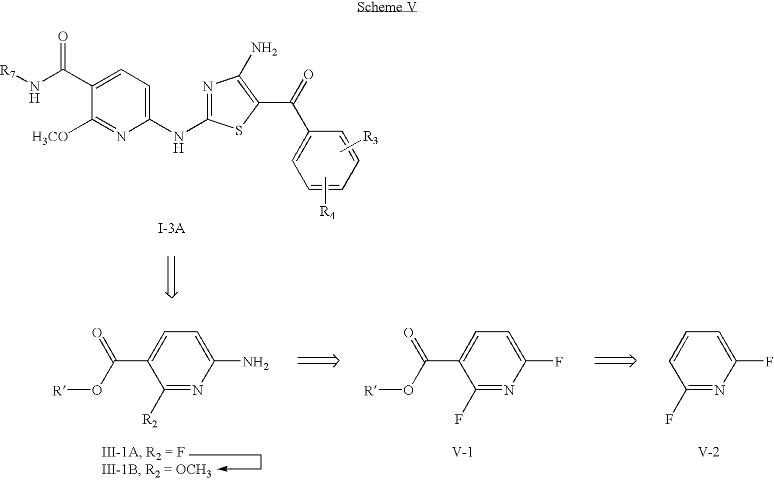

As shown in Scheme VI below, a tetrasubstituted pyridine such as 1-3C is accessible by building upon the trisubstitution sequence above. The anion regioselectively formed from 2-chloro-6-methoxy-pyridine (VI-1) in Comins, D. L.; Baevsky, M. F.; Hong, H. J. Am. Chem. Soc. 1992, 114, 10971-10972—can be acylated with a chloroformate, carbon dioxide, or di-tertbutyl dicarbonate to give nicotinate VI-2, which in turn can be transformed to the amide VI-3 via acid chloride or through carboxylate and amine coupling. Another deprotonation directed by the amide in VI-3 can give an anion that can be capped—with an electrophile, such as a halogen source (iodine, N-chlorosuccinimide, or N-bromosuccinmide), exemplified in this example with iodine, to furnish the tetrasubstituted pyridine VI-4. Such a sequence to achieve tetrasubstitution was accomplished in Comins, D. L.; Baevsky, M. F.; Hong, H. J. Am. Chem. Soc. 1992, 114, 10971-10972. Selective replacement of the choride in VI-4 with amine would provide requisite precursor III-1C.

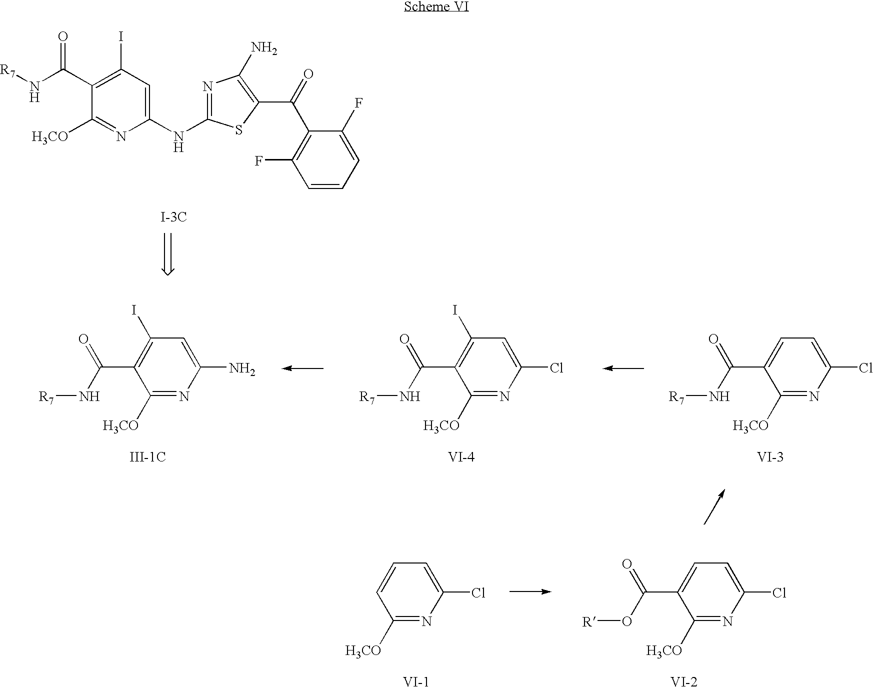

As shown below in Scheme VII, other pyridine substitution patterns can be produced from those preexisting and purchasable, after simple manipulation: commercially available 2,6-dichloro-4-methyl-nicotinonitrile (VII-1) is selectively substituted by ammonia at the 2-position to afford monochloride VII-2, see Katritzky, A. R.; Rachwal, S.; Smith, T. P.; Steel, P. J. J. Heterocycl. Chem. 1995, 32, 979-984 and references therein. Chloride VII-2 is a very versatile intermediate, if the chloride is removed by reduction, the nitrile VII-3 results, which can be hydrolyzed to the corresponding acid VII-4, and processed to the target I-3D along the route in Scheme III. Alternatively, the intermediate VII-2 can be directly hydrolyzed to the chloro-acid VII-7, which according to Scheme III, leads eventually to target I-3E. Another option is solvolysis of the chloride in VII-2 with methanol to the methoxy VII-5, and similar processing via carboxyl VII-6 would provide target I-3F. Likewise, the pathway through intermediate chloride VII-7 could furnish 1) through removal of chlorine the target I-3D, 2) via displacement with a nucleophile to the ether target I-3F.

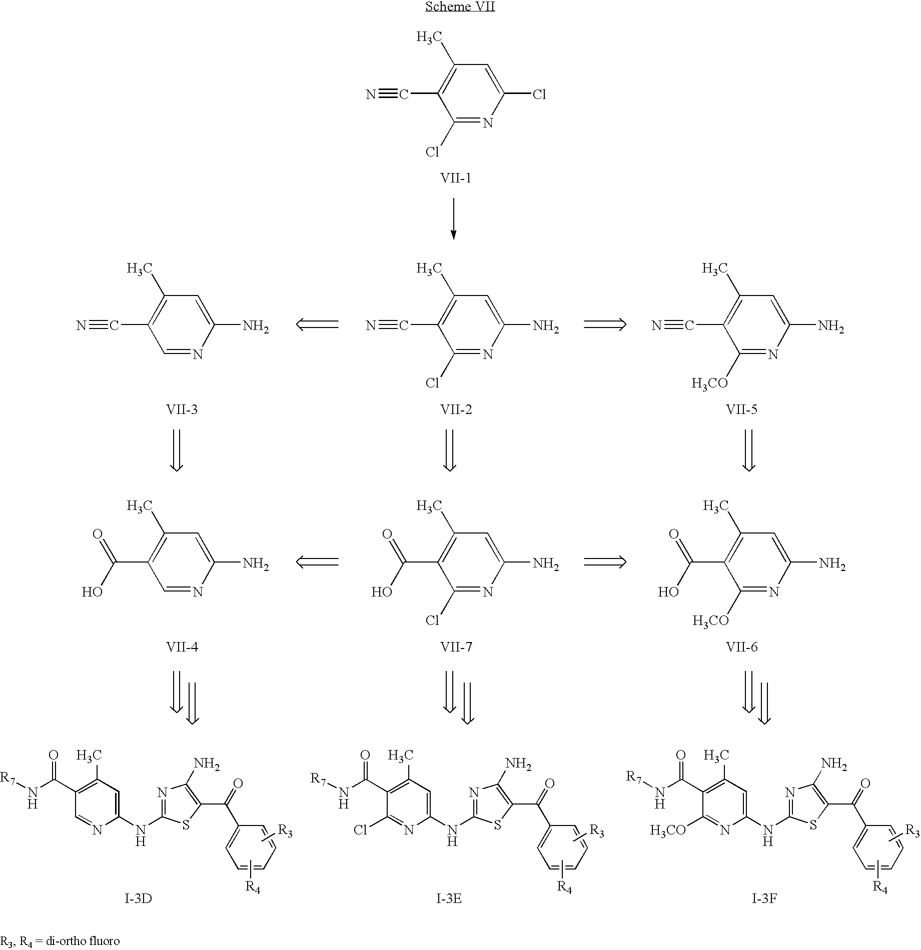

As depicted in Scheme VIII, the diester 111-1G leads to additional diversity in the substituents on the pyridine ring. According to Cobo, J.; Garcia, C.; Melguizo, M.; Sanchez, A.; Nogueras, M. Tetrahedron 1994, 50, 10345-10358, the pyridine ring is produced after a Diels-Alder cycloaddition with dimethyl acetylenedicarboxylate (VIII-1) and pyrimidinone VIII-2. The 2-amine of III-1G may have to be temporarily protected as a pivalate, as seen for example, in Hirokawa, Y.; Yoshida, N.; Kato, S. Bioorg. Med. Chem. Lett. 1998, 8, 1551-1554, but the diester III-1G can be hydrolyzed to its corresponding diacid, converted to a phthalic anhydride, and treated with an amine to furnish amide-carboxylate I-3G. If this amidation sequence is done twice, by orchestrating the order of amine and ammonia, a diamide analog I-3H can be obtained. If the carboxy-amide I-3G is heated and/or dehydrated, the imide analog I-3J can be produced.

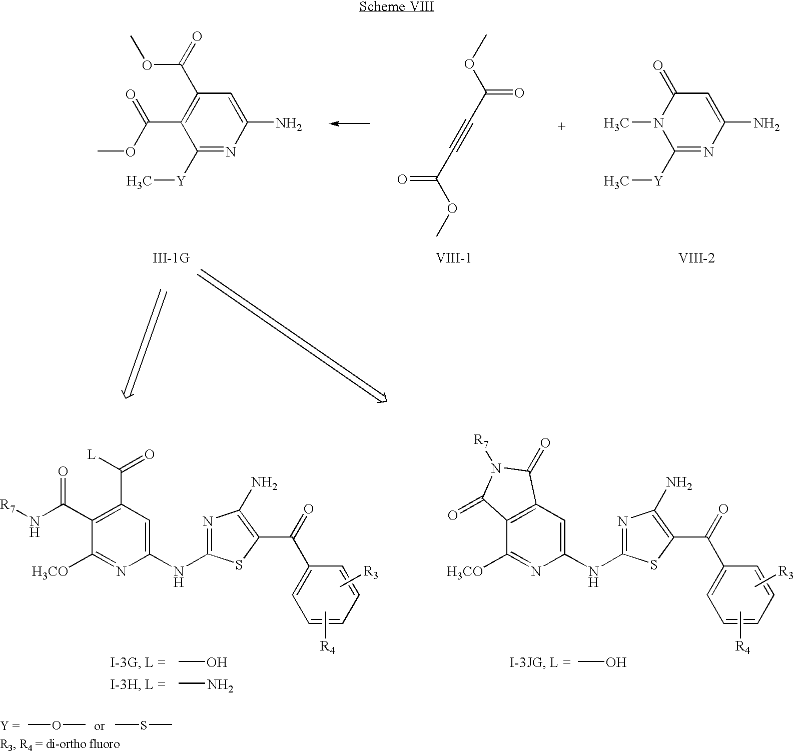

Many variants with carboxyl derivatives are heretofore described, and activity can accompany sulfonamides, and preferably N-monoalkyl derivatives, as depicted by examples IX-1 in Scheme IX below. The hydrogen on nitrogen of sulfonamides such as IX-1—are fairly acidic and can interfere in synthesis, as will be shown below—and can be protected as shown in structure IX-2. Specific examples of protection are discussed below.

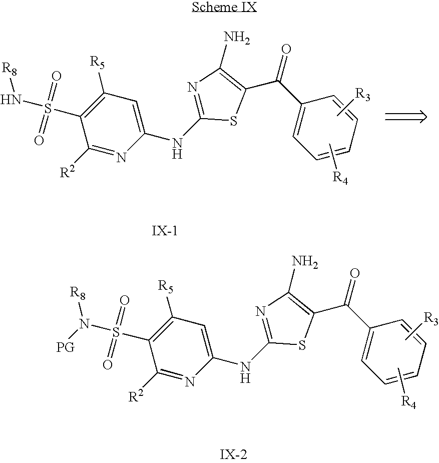

A suggested protecting group, N-2,4-dimethoxybenzyls is portrayed in Scheme X below. The final deprotection of IX-2A to N-mono-alkyl IX-1 can be accomplished under numerous conditions (see Greene, T. W.; Wuts, P. G. M. In Protective Groups in Organic Synthesis; Third ed.; 1999, 640), that will accommodate most functional groups. The preparation of substrate IX-2A starts at the bottom of Scheme X, from the primary amine X-6, which is reductively alkylated With 2,4-dimethoxy-benzaldehyde to benzylamine X-5. Condensation with 2-chloropyridine-5-sulfonyl chlorides (X-4; for R₂=R₅=—H, see Naegeli, C.; Kündig, W.; Brandenburger, H. Helv. Chim. Acta 1939, 21, 1746-1756) to 2-chloro-5-sulfonamide X-2 and subsequent solvolysis with ammonia provides 2-amino X-3. As discussed for Scheme III, amines such as X-3 can be transformed to the corresponding isothiocyanates X-1, which in turn can be processed via Scheme II to the desired 2,4-diaminothiazoles IX-2A.

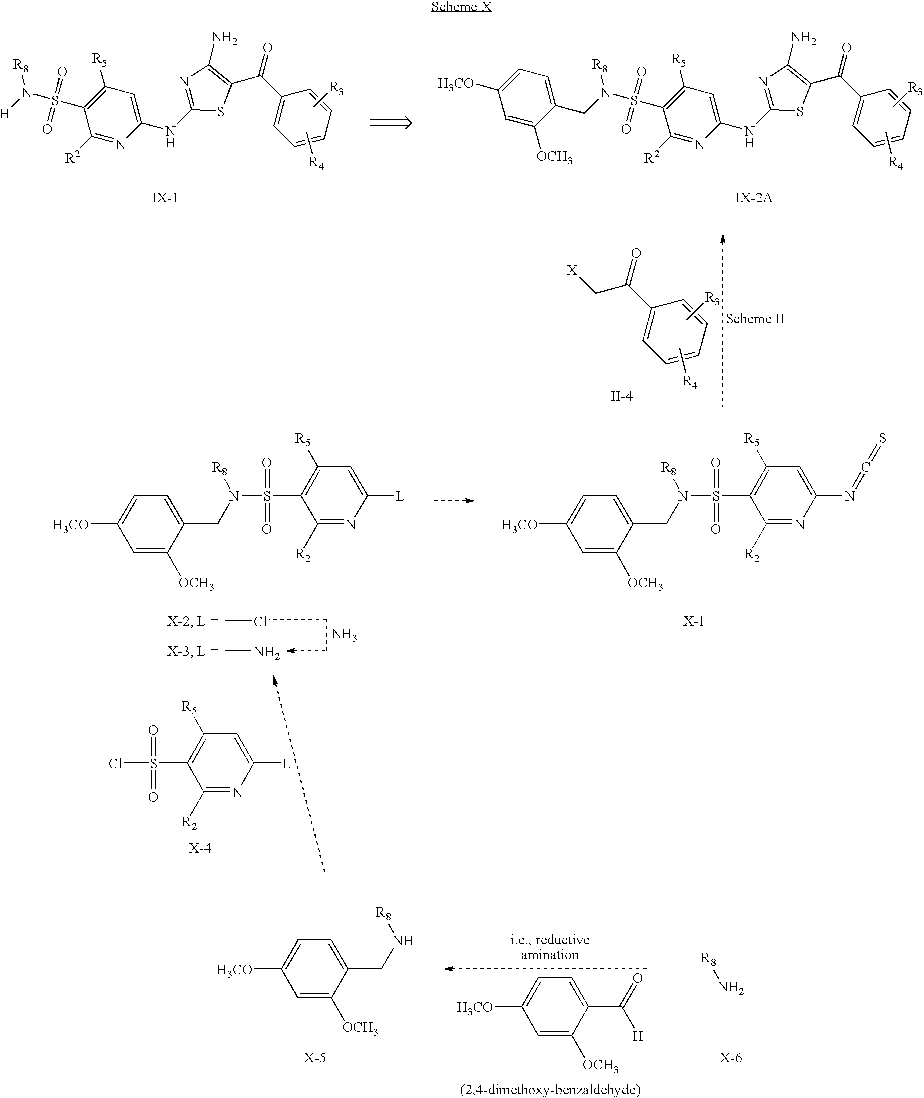

For the particular cases when primary sulfonamides are desired, as shown in Scheme XI below with targets X-1B, similar protection via the N-(2,4-dimethoxybenzylidene) X-2B, which can be liberated under acidic conditions. At the bottom of Scheme XI, the 2-chloro-5-sulfonamide XI-4 can be condensed with 2,4-dimethoxybenzaldehyde to imine XI-2, which undergoes substitution to 2-amino XI-3. Subsequent formation of the corresponding isothiocyanate XI-1 according to the discussion for Scheme III and further processing via Scheme II provides protected thiazoles IX-2B.

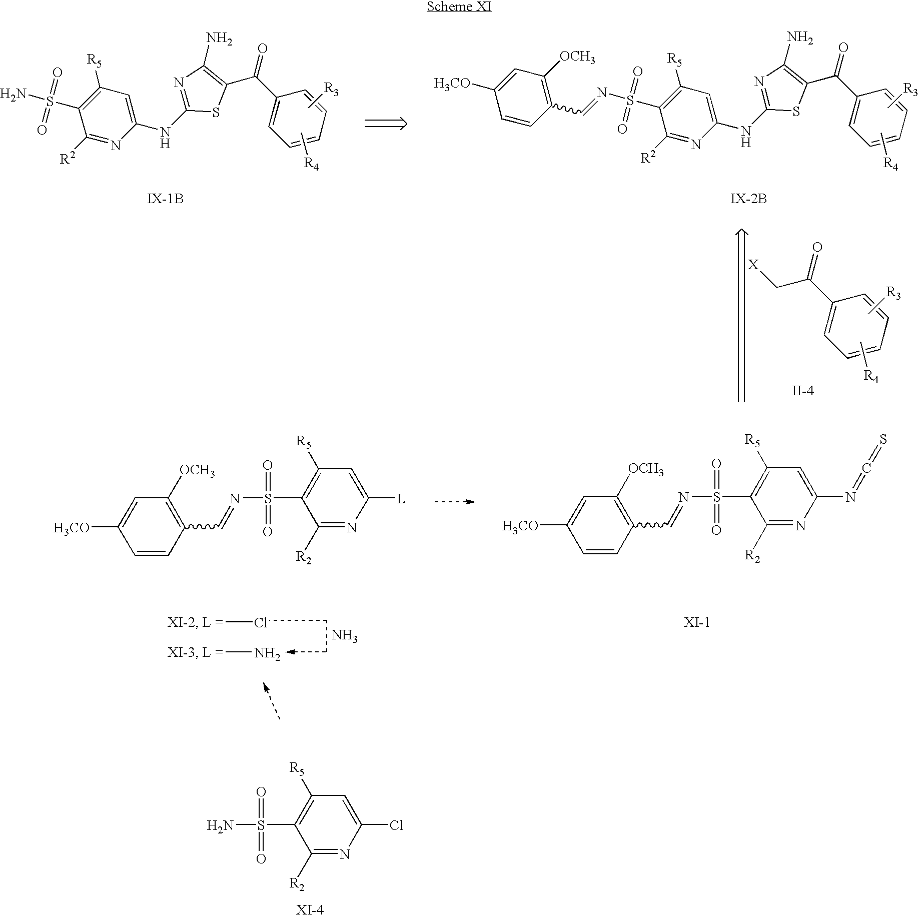

Another viable approach is outlined in Scheme XII below. The 2-tritylaminothiazole is made according to analogous precedent of World Patent Application WO 00/75120. Trifluoroacetic acid liberates the 2,4-diamine XII-1, that undergoes alkylation with selectivity reported in the aforementioned patent application to substitute the pyridine activated at the 2-position as in X-2A to afford the desired IX-2C, which can be processed as outlined in Scheme IX.

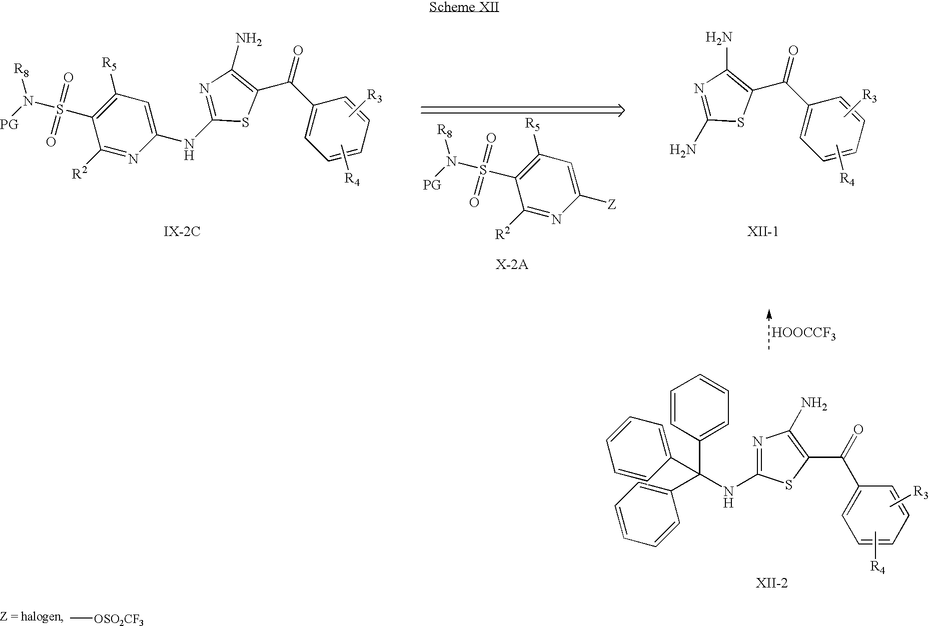

### Example 1A

### 6-[4-Amino-5-(2,6-difluoro-benzoyl)-thiazol-2-ylamino]-N-((N-methyl-pyrrolidin-2S-ylmethyl)-nicotinamide

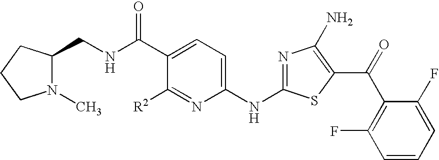

The title compound was prepared as follows: to a solution of 6-[4-amino-5-(2,6-difluoro-benzoyl)-thiazol-2-ylamino]-nicotinic acid (0.210 g, 0.560 mmol) in N,N-dimethylformamide (DMF; 3 mL) was added in succession triethylamine (0.156 mL, 1.12 mmol), O-(7-azabenzotriazol-1-yl)-1,1,3,3-tetramethyluronium hexafluorophosphate (HATU; 0.255 g, 0.67 mmol commercially available from Perspective Biosystems), and (N-methyl-pyrrolidin-2S-yl)-methylamine (0.128 g, 1.12 mmol; Sassaman, M. B.; Giovanelli, J.; Sood, V. K.; Eckelman, W. C. Bioorg. Med. Chem. 1998, 6, 1759-1766). After 18 h, the resulting solution was concentrated in vacuo to a brown residue and purified via flash column chromatography with 1% (58% NH₄OH) in 10% MeOH/CHCl₃ as eluant to give a yellow powder in 60% yield.

¹H NMR (DMSO-d₆): δ 8.60 (d, 1H, J=1.7 Hz), 7.92 (dd, 1H, J=2.1, 8.7 Hz), 7.50-7.35 (m, 1H), 7.12 (dd, 2H, J=7.7, 8.1 Hz), 2.16 (s, 3H).

LC-MS (M+H⁺): 473.

Anal. Calcd. for C₂₂H₂₂F₂N₆O₂S.0.8H₂O: C, 54.27; H, 4.89; N, 17.26; S, 6.59. Found: C, 54.01; H; 5.16; N, 17.09; S, 6.25.

The starting materials for above were prepared as follows:
6-Amino-nicotinic Acid Ethyl Ester

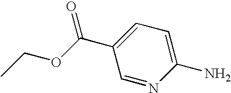

HCl gas was passed through a solution of 6-amino-nicotinic acid (2.00 g, 14.5 mmol) in ethanol (40 mL) at 0° C. for 10 min, then warmed to reflux. After 18 h, the mixture was allowed to cool, concentrated in vacuo to a colorless solid, which was partitioned with EtOAc and sat. aq. Na₂CO₃. The aqueous layer was separated and extracted with EtOAc. The combined organic layers were dried over Na₂SO₄ and evaporated. Recrystallization in EtOH gave 2.20 g (92% yield) of white solid that was typically used without further purification.

¹H NMR (CDCl₃): □8.75 (d, 1H, J=2.1 Hz), 8.07 (dd, 1H, J=8.7, 2.1 Hz), 6.51 (d, 1H, J=8.7 Hz), 4.96 (bs, 2H), 4.35 (q, 2H, J=7.2 Hz), 1.40 (t, 3H, J=7.2 Hz).
6-Isothiocyanato-nicotinic Acid Ethyl Ester

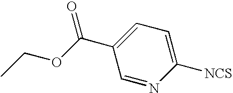

A solution of thiophosgene (1.10 mL, 14.4 mmol) in acetone (20 mL) and sat. aq. NaHCO₃ (40 mL) were both added simultaneously to a suspension of 6-amino-nicotinic acid ethyl ester (2.00 g, 12.0 mmol) in acetone (20 mL) and CHCl₃ (20 mL) at −10° C. (salt-ice bath)—at such a rate that the internal temperature did not exceed 5° C. Subsequently the acetone and chloroform were removed under reduced pressure. The resultant residue was extracted with CH₂Cl₂, concentrated, and purified with flash column using CH₂Cl₂ as eluant to give 1.54 g (61% yield) of orange crystals, which were used without any further purification.

¹H NMR (CDCl₃): □8.96 (d, 1H, J=2.2 Hz), 8.24 (dd, 1H, J=2.2, 8.3 Hz), 7.07 (d, 1H, J=8.3 Hz), 4.34 (q, 2H, J=7.2 Hz), 1.34 (t, 3H, J=7.2 Hz).
2-Bromo-2′,6′-difluoroacetophenone

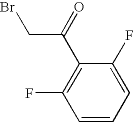

To a mechanically stirring solution of 2′,6′-difluoroacetophenone (100.0 g, 640.0 mmol; Melford Laboratories, Ltd.) in ethyl acetate (1300 mL) was added freshly milled copper(II) bromide (300 g, 1.35 mol) and bromine (1.6 mL, 32 mmol). The mixture was heated at reflux for 2.25 hours and allowed to cool to ambient temperature. The resultant green mixture was filtered and the solids rinsed with ethyl acetate (4×100 mL). The filtrate was concentrated with a rotary evaporator at <40° C. under reduced pressure, diluted with methyl t-butyl ether (MTBE; 650 mL), filtered through a pad of silica gel (230-400 □; 9.5 cm diam.×4 cm. ht.), and the solids rinsed with MTBE (5×200 mL). Concentration of the filtrate gave a pale green oil, which was purified by fractional vacuum distillation to give 117 g (78% yield) of 2-bromo-2′,6′-difluoroacetophenone as a pale yellow oil, bp 88-97° C. (2.0 mm Hg). The compound matched that previously described in World Patent Application WO99/21845 and was typically used without any further purification or characterization.

¹H NMR(CDCl₃): □7.48 (ddd, 1H, J=6.3, 8.5, 14.8 Hz), 7.01 (ddd, 2H, J=4.6, 5.8, 16.6 Hz), 4.37 (t, 2H, J=0.7 Hz).
2-[4-Amino-5-(2,6-difluoro-benzoyl)-thiazol-2-ylamino]-pyridine-5-carboxylic Acid Ethyl Ester

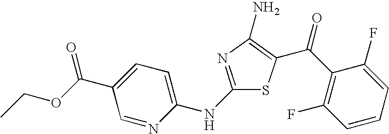

To a solution of 6-isothiocyanato-nicotinic acid ethyl ester (300 mg, 1.44 mmol) in CH₃CN (5 mL) was added cyanamide (67 mg, 1.6 mmol) and 1,8-diazabicyclo[5.4.0]-undec-7-ene (DBU; 0.259 mL, 1.72 mmol). After 0.5 h, 2-bromo-2′,6′-difluoroacetophenone (0.355 g, 1.51 mmol) and DBU (0.259 mL, 1.72 mmol) were added. After an additional 1.5 h, the solution was diluted with 10% MeOH/CHCl₃ (100 mL), washed with H₂O (25 mL×2), dried over Na₂SO₄, and concentrated to a brown solid, which was purified via column chromatography with 1% (58% NH₄OH) in 15% MeOH/CHCl₃ as eluant to give 161 mg (40% yield) of a brown powder, which was used without any further purification.

¹H NMR (DMSO-d₆): 58.78 (d, 1H, J=2.0 Hz), 8.21 (dd, 1H, J=2.2, 8.7 Hz), 8.00 (bs, 2H), 7.62-7.52 (m, 1H), 7.22 (dd, 2H, J=7.7, 8.1 Hz), 7.19 (d, 1H, J=8.8 Hz), 4.28 (q, 2H, J=7.1 Hz), 1.28 (t, 3H, J=7.1 Hz).
6-[4-Amino-5-(2,6-difluoro-benzoyl)-thiazol-2-ylamino]-nicotinic Acid

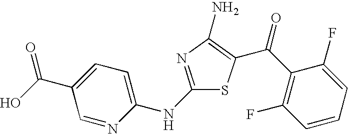

To a suspension of 6-[4-amino-5-(2,6-difluoro-benzoyl)-thiazol-2-ylamino]-nicotinic acid ethyl ester (0.29 g, 0.72 mmol) in MeOH (3 mL) was added 3N NaOH (2.4 mL, 7.2 mmol). The resulting solution was stirred at ambient temperature for 18 h. The methanol was removed in vacuo, and the resultant solution was brought to pH 3 with 10% aq. HCl. The precipitate was filtered off and dried to give 259 mg (96% yield) of a yellow solid, which was used without any further purification.

¹H NMR (DMSO-d₆): δ 12.37 (s, 1H), 8.75 (s, 1H), 8.02 (dd, 1H, J=2.1, 8.7 Hz), 8.01 (bs, 2H), 7.62-7.51 (m, 1H), 7.30-7.12 (m, 3H).

### Example 1B

### 6-[4-Amino-542,6-difluoro-benzoyl)-thiazol-2-ylamino]-N-(1-dimethylamino-cyclobutylmethyl)-nicotinamide

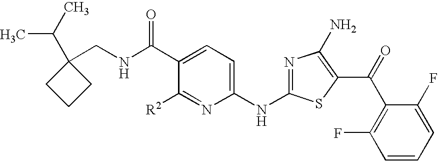

The title compound was prepared in a manner analogous to that used for Example 1A. 1-Aminomethyl-1-dimethylaminocyclobutane (Yang, D.; Brémont, B.; Shen, S.; Kefi, S.; Langlois, M. Eur. J. Med. Chem. Chim. Ther. 1996, 31, 231-240) and 6-[4-amino-5-(2,6-difluoro-benzoyl)-thiazol-2-ylamino]-nicotinic acid from Example 1A provided a yellow solid in 62% yield.

¹H NMR (DMSO-d₆): δ8.75 (s, 1H), 8.30 (bs, 1H), 8.15 (d, 1H, J=8.0 Hz), 7.60-7.51 (m, 1H), 7.25 (dd, 2H, J=7.6, 8.3 Hz), 7.14 (d, 1H, J=8.7 Hz), 3.50 (bd, 2H, J=5.3 Hz), 2.19 (bs, 6H).

HRMS: Calcd. for C₂₃H₂₅F₂N₆O₂S (M+H⁺): 487.1746. Found: 487.1728.

Anal. Calcd. for C₂₃H₂₄F₂N₆O₂S.1.2H₂O: C, 54.36; H, 5.24; N, 16.54; S, 6.31. Found: C, 54.33; H, 5.28; N, 16.22; S, 6.14.

### Example 1C

### 6-[4-Amino-5-(2,6-difluoro-benzoyl)-thiazol-2-ylamino]-N-pyridin-2-ylmethyl-nicotinamide

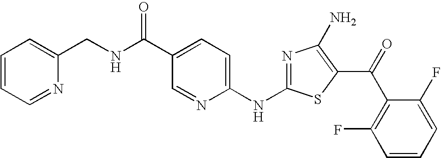

The title compound was prepared in a manner analogous to that used for Example 1A. 2-(Aminomethyl)-pyridine and 6-[4-amino-5-(2,6-difluoro-benzoyl)-thiazol-2-ylamino]-nicotinic acid from Example 1A provided a yellow solid in 32% yield.

¹H NMR (DMSO-d₅): □ 12.31 (s, 1H), 9.13 (dd, 1H, J=6.8, 7.0 Hz), 8.88 (d, 1H, J=1.9 Hz), 8.53 (d, 1H, J=4.1 Hz), 8.23 (dd, 1H, J=1.9, 8.3 Hz), 8.01 (bm, 1H), 7.75 (ddd, 1H, J=2.3, 7.9, 8.3 Hz), 7.62 (ddd, 1H, J=8.7, 8.7, 15.8 Hz), 4.51 (d, 2H, J=5.6 Hz).

LC-MS (M+H⁺): 467.

Anal. Calcd. for C₂₂H₁₆F₂N₆O₂S.0.3H₂O: C, 55.95; H, 3.75; N, 17.17; S, 6.55. Found: C, 55.90; H, 3.82; N, 17.10; S, 6.37.

### Example 1D

### 6-[4-Amino-5-(2,6-difluoro-benzoyl)-thiazol-2-ylamino]-N-pyridin-3-ylmethyl-nicotinamide

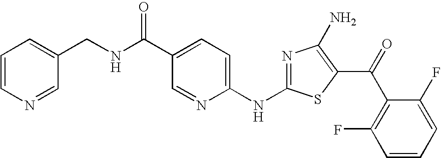

The title compound was prepared in a manner analogous to that used for Example 1A. 3-(Aminomethyl)-pyridine and 6-[4-amino-5-(2,6-difluoro-benzoyl)-thiazol-2-ylamino]-nicotinic acid from Example 1A provided a yellow solid in 37% yield.

¹H NMR (DMSO-d₆): □ 12.00 (s, 1H), 9.18 (dd, 1H, J=5.7, 6.0 Hz), 8.80 (s, 1H), 8.60 (s, 1H), 8.51 (d, 1H, J=5.8 Hz), 8.20 (s, 1H), 8.00 (bs, 2H), 7.72 (d, 1H, J=7.7 Hz), 7.51-7.49 (m, 1H), 7.12 (dd, 1H, J=4.7, 7.9 Hz), 7.32 (dd, 3H, J=7.9, 7.9 Hz), 7.01 (dd, 1H, J=8.7 Hz), 4.50 (d, 2H, J=5.8 Hz). LC-MS (M+H⁺): 467.

Anal. Calcd. for C₂₂H₁₆F₂N₆O₂S.1.3H₂O.0.2CHCl₃: C, 51.90; H, 3.69; N, 16.36; S, 6.24. Found: C, 51.81; H, 4.00; N, 16.39; S, 6.19.

### Example 1E

### 6-[4-Amino-5-(2,6-difluoro-benzoyl)-thiazol-2-ylamino]-N-phenyl-nicotinamide

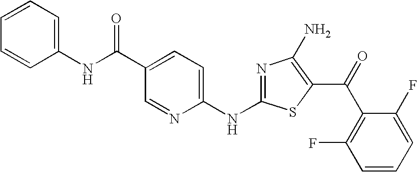

The title compound was prepared in a manner analogous to that used for Example 1A. Aniline and 6-[4-amino-5-(2,6-difluoro-benzoyl)-thiazol-2-ylamino]-nicotinic acid from Example 1A provided a yellow solid in 37% yield.

¹H NMR (DMSO-d₆): □ 12.26 (s, 1H), 10.12 (s, 1H), 8.93 (d, 1H, J=2.1 Hz), 8.26 (dd, 1H, J=1.9, 8.5 Hz), 8.10-7.91 (m, 2H), 7.62 (d, 2H, J=7.9 Hz), 7.52-7.51 (1H, m), 7.41-7.15 (m, 5H), 7.10 (dd, 1H, J=7.2, 7.5 Hz).

LC-MS (M+H⁺): 452.

Anal. Calcd. for C₂₂H₁₅F₂N₅O₂S.0.5MeOH.0.2 NH₄OH: C, 56.98; H, 3.78; N, 15.36; S, 6.76. Found: C, 56.94; H, 3.96; N, 15.61; S, 6.52.

### Example 1F

### 6-[4-Amino-5-(2,6-difluoro-benzoyl)-thiazol-2-ylamino]-N-(2-piperidin-1-yl-ethyl)-nicotinamide

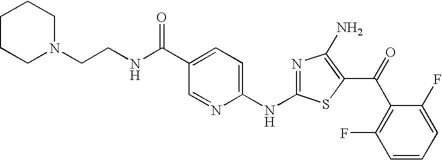

The title compound was prepared in a manner analogous to that used for Example 1A. 1-(2-Aminoethyl)-piperidine and 6-[4-amino-5-(2,6-difluoro-benzoyl)-thiazol-2-ylamino]-nicotinic acid from Example 1A provided a yellow solid in 57% yield.

¹H NMR (CD₃OD): □ 8.64 (d, 1H, J=1.7 Hz), 8.06 (dd, 1H, J=2.4, 8.7 Hz), 7.51-7.40 (m, 1H), 7.05-6.90 (m, 3H), 3.49 (dd, 2H, J=6.7, 7.0 Hz).

LC-MS (M+H⁺): 452.

Anal. Calcd. for C₂₄H₂₅F₂N₅O₂S.0.5CHCl₃.1.0NH₄OH: C, 50.71; H, 5.30; N, 14.48; S, 5.53. Found: C, 50.72; H, 5.04; N, 14.25; S, 5.38.

### Example 1G

### 6-[4-Amino-52,6-difluoro-benzoyl)-thiazol-2-ylamino]-N-pyridin-2-yl-nicotinamide

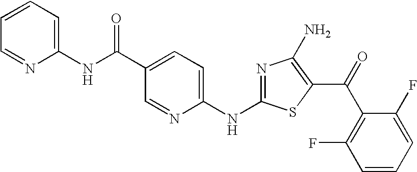

The title compound was prepared in a manner analogous to that used for Example 1A. 2-Aminopyridine and 6-[4-amino-5-(2,6-difluoro-benzoyl)-thiazol-2-ylamino]-nicotinic acid from Example 1A provided a yellow solid in 28% yield.

¹H NMR (DMSO-d₆): δ 12.43 (s, 1H), 10.97 (s, 1H), 9.12 (d, 1H, J=2.1 Hz), 8.56 (d, 1H, J=4.0 Hz), 8.48 (dd, 1H, J=2.1, 8.7 Hz), 8.35 (d, 1H, J=8.3 Hz), 8.25-8.15 (bs, 2H), 8.05 (ddd, 1H, J=1.7, 6.8, 15.6 Hz), 7.85-7.71 (m, 1H), 7.50-7.30 (m, 4H).

LC-MS (M+H⁺): 453.

Anal. Calcd. for C₂₁H₁₄F₂N₆O₂S.0.2H₂O: C, 55.31; H, 3.18, N, 18.43; S, 7.03. Found: C, 54.99; H, 3.29; N, 18.35; S, 6.97.

### Example 1H

### 6-[4-Amino-5-(2,6-difluoro-benzoyl)-thiazol-2-ylamino]-N-(1-ethyl-pyrrolidin-2-ylmethyl)-nicotinamide

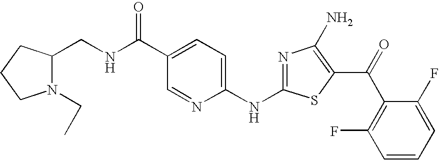

The title compound was prepared in a manner analogous to that used for Example 1A. 2RS-Aminomethyl-N-ethyl-pyrrolidine and 6-[4-amino-5-(2,6-difluoro-benzoyl)-thiazol-2-ylamino]-nicotinic acid from Example 1A provided a yellow solid in 77% yield.

¹H NMR (DMSO-d₆): □ 8.60 (d, 1H, J=2.1 Hz), 8.23 (dd, 1H, J=5.7, 5.9 Hz), 7.97 (dd, 1H, J=2.1, 8.5 Hz), 7.87-7.75 (m, 2H), 7.50-7.38 (m, 1H), 7.14 (dd, 2H, J=7.9, 7.9 Hz), 7.02 (d, 1H, J=8.7 Hz), 0.89 (t, 3H, J=7.0 Hz).

LC-MS (M+H⁺): 487.

Anal. Calcd. for C₂₃H₂₄F₂N₆O₂S.1.5H₂O: C, 53.79; H, 5.30; N, 16.36; S, 6.24. Found: C, 53.50; H, 5.54; N, 16.65; S, 5.84.

### Example 1I

### 6-[4-Amino-5-(2,6-difluoro-benzoyl)-thiazol-2-ylamino]-N-(2-dimethylamino-1R-methyl-ethyl)-nicotinamide

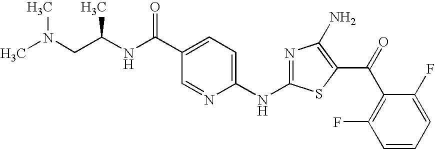

The title compound was prepared in a manner analogous to that used for Example 1A. 1-Dimethylaminoprop-2S-yl-amine bis-toluenesulfonic acid salt and 6-[4-amino-5-(2,6-difluoro-benzoyl)-thiazol-2-ylamino]-nicotinic acid from Example 1A provided a yellow solid in 70% yield.

¹H NMR (DMSO-d₆): □ 8.70 (d, 1H, J=2.1 Hz), 8.07 (d, 2H, J=8.3 Hz), 7.95-7.92 (m, 2H), 7.55-7.45 (m, 1H), 7.20 (dd, 1H, J=7.7, 8.1 Hz), 7.05 (d, 1H, J=8.7 Hz), 4.10-4.05 (m, 1H), 1.05 (d, 3H, J=6.6 Hz).

LC-MS (M+H⁺): 461. Anal. Calcd. for C₂₁H₂₂F₂N₆O₂S.1.5H₂O: C, 51.74; H, 5.17; N, 17.24; S, 6.58. Found: C, 52.04; H, 5.17; N, 17.29; S, 6.55.

The starting material for the above was available as follows:
N-Benzyloxycarbonyl-D-alanine Dimethylamide 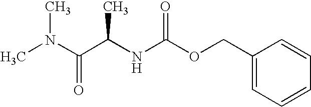
N-Benzyloxycarbonyl-D-alanine (10.4 g; 46.6 mmol) and N-hydroxy-benzotriazole hydrate (HOBt.H₂O; 6.30 g; 46.6 mmol) were suspended in CH₃CN (185 mL) at ambient temperature. The suspension was cooled to −3° C., and then a solution of dicyclohexylcarbodiimide (9.61 g; 46.6 mmol) in CH₃CN (21 mL) was added dropwise while maintaining the internal temperature below 2° C. After stirring at 0° C. for 2.5 h, dimethylamine hydrochloride (5.70 g; 69.9 mmol) was added. Diisopropylethylamine (24.3 mL; 140 mmol) was added dropwise while maintaining the internal temperature below 4° C. The suspension was stirred at 0° C. for 1.5 h, then was allowed to warm to ambient temperature and stir overnight (16 h). The suspension was concentrated to give a white solid, which was slurried in EtOAc (300 mL) and then vacuum-filtered through a pad of Celite. The filtrate was washed with 1N HCl (310 mL), 5% aqueous Na₂CO₃ (2×210 mL), and saturated aqueous NaCl (105 mL), and then dried over MgSO₄, filtered, and concentrated to give an oil/solid mixture. The residue was suspended in MTBE (210 mL) and stirred at ambient temperature for 24 h. The suspension was vacuum-filtered through a pad of Celite, and the filtrate was concentrated to give 9.3 g (79% yield) of a slightly cloudy pale yellow oil, which displayed a ¹H NMR (CDCl₃) spectrum that matched literature (Isogai, A.; Suzuki, A.; Tamura, S.; Higashikawa, S.; Kuyama, S. J. Chem. Soc., Perkin Trans. 1 1984, 1405-1411), and was used without any further purification.

¹H NMR (DMSO-d₆): □ 7.43 (bd, 1H, J=7.7 Hz), 7.27-7.40 (m, 5H), 5.01 (s, 2H), 4.49 (app pentet, 1H, J=7.2 Hz), 3.00 (s, 3H), 2.82 (s, 3H), 1.16 (d, 3H, J=7.0 Hz).

MS-APCI (M+H⁺) 251, (M−NMe₂+H⁺) 207, (M-Cbz+2H+)117.
D-Alanine Dimethylamide

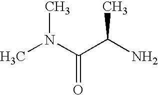

A mixture of N-benzyloxycarbonyl-D-alanine dimethylamide (10.6 g; 40.0 mmol), 10% palladium on carbon (1.1 g) and anhydrous ethanol (210 mL) was hydrogenated with a Parr shaker (˜45 p.s.i. H₂). The mixture was vacuum-filtered through Celite, and the filtrate was concentrated to give 5.22 g (106% yield) of an oil that was contaminated with 8 mol % ethanol, displayed a ¹H NMR (CDCl₃) spectrum that matched literature (for DL-alanine dimethylamide; Guthrie, R. D.; Hrovat, D. A.; Prahl, F. G.; Swan, J. J. Org. Chem. 1981, 46, 498-501), and used without further purification.

¹H NMR (DMSO-d₆): □ 3.83 (app q, 1H, J=6.8 Hz), 3.76 (bs, 2H), 2.97 (s, 3H), 2.81 (s, 3H), 1.09 (d, 3H, J=6.8 Hz).
1-Dimethylaminoprop-2R-yl-amine bis-Toluenesulfonic Acid Salt

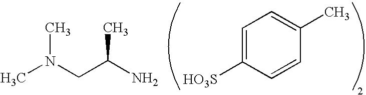

A solution of D-alanine dimethylamide (5.07 g; 43.6 mmol) in dry THF (175 mL) was added dropwise to a solution of LiAlH₄ in THF (92 mL of a 1.0 M solution; 92 mmol). The resulting cloudy solution was heated at reflux overnight (17 h). H₂O (3.5 mL) was carefully added to the mixture. After stirring the suspension for 0.5 h, 15% aqueous NaOH (3.5 mL) was carefully added. After stirring for an additional 0.5 h, H₂O (10.4 mL) was added. The suspension was stirred at 23° C. for 1 h, and was then vacuum-filtered. The salts were rinsed with THF (2×50 mL). p-Toluenesulfonic acid hydrate (17.4 g; 91.5 mmol) was added to the filtrate. The resulting suspension was concentrated to a volume of ˜50 mL and was then filtered to give 3.6 g (18% yield) of a white solid. Additional product was recovered as follows: The filtrate was concentrated to give a wet solid, which was then dissolved in anhydrous ethanol (50 mL) and again concentrated. The dissolution/concentration process was repeated once more. The resulting solid was then suspended in THF (100 mL) and vacuum-filtered to give additional product, 5.3 g (27% yield) of a white solid. The overall total yield was 45%. The solid was stored under anhydrous conditions and typically used without any further purification.

¹H NMR (CD₃OD): □ 7.73 (d, 4H, J=8.2 Hz), 7.27 (d, 4H, J=8.0 Hz), 3.89 (X portion of ABX, 1H, app sextet, J=6.5 Hz), 3.44 (AB portion of ABX, 2H, J_{AX}=5.8 Hz, J_{BX}=2.2 Hz, □□_{AB}=20.0 Hz), 3.01 (bs, 6H), 2.39 (s, 6H), 1.46 (d, 3H, J=6.7 Hz).

MS-APCI: (M−2TsOH+H⁺) 103.

### Example 1J

### 6-[4-Amino-5-(2,6-difluoro-benzoyl)-thiazol-2-ylamino]-N-(2-pyrrolidin-1-yl-ethyl)-nicotinamide

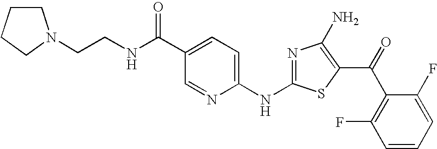

The title compound was prepared in a manner analogous to that used for Example 1A. 1-(2-Aminoethyl)-pyrrolidine and 6-[4-amino-5-(2,6-difluoro-benzoyl)-thiazol-2-ylamino]-nicotinic acid from Example 1A provided a yellow solid in 65% yield.

¹H NMR (DMSO-d₆): □8.72 (d, 1H, J=1.9 Hz), 8.42 (dd, 1H, J=5.5, 5.7 Hz), 8.10 (dd, 1H, J=2.3, 8.5 Hz), 7.95-7.90 (m, 2H), 7.52-7.41 (m, 1H), 7.20 (dd, 1H, J=7.5, 8.1 Hz), 6.98 (d, 1H, J=8.5 Hz), 1.61-1.42 (m, 4H).

LC-MS (M+H⁺): 473.

Anal. Calcd. for C₂₂H₂₂F₂N₈O₂S.1.2H₂O: C, 53.48; H, 4.98; N, 17.01; S, 6.49. Found: C, 53.54; H, 4.99; N, 17.19; S, 6.39.

### Example 1K

### 6-[4-Amino-5-(2,6-difluoro-benzoyl)-thiazol-2-ylamino]-N-(2-dimethylamino-ethyl)-nicotinamide

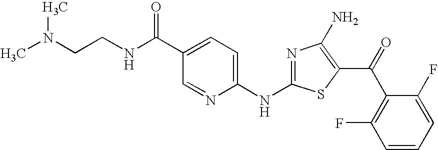

The title compound was prepared in a manner analogous to that used for Example 1A. N,N-Dimethylethylenediamine and 6-[4-amino-5-(2,6-difluoro-benzoyl)-thiazol-2-ylamino]-nicotinic acid from Example 1A provided a yellow solid in 77% yield.

¹H NMR (CD₃OD): □ 8.75 (d, 1H, J=2.3 Hz), 8.17 (dd, 1H, J=2.1, 8.7 Hz), 7.51-7.42 (m, 1H), 7.10-7.00 (m, 3H), 3.52 (dd, 2H, J=6.4, 6.6 Hz), 2.49 (s, 6H).

LC-MS (M+H⁺): 447. Anal. Calcd. for C₂₀H₂₀F₂N₆O₂S.1.9H₂O.3 EtOH: C, 50.03; H, 5.22; N, 16.99; S, 6.48. Found: C, 50.26; H, 5.00; N, 16.98; S, 6.18.

### Example 1L

### 6-[4-Amino-5-(2,6-difluoro-benzoyl)-thiazol-2-ylamino]-N-2-dimethylamino-2-methyl-propyl)-nicotinamide

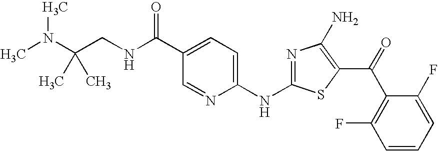

The title compound was prepared in a manner analogous to that used in Example 1A. 6-[4-Amino-5-(2,6-difluoro-benzoyl)-thiazol-2-ylamino]-nicotinic acid (0.400 g, 1.06 mmol) and 2, N2,N2-trimethyl-propane-1,2-diamine hydrochloride (469 mg, 2.12 mmol) provided a yellow solid in 69% yield.

¹H NMR (DMSO-d₆): δ 12.17 (bs, 1H), 8.78 (d, 1H, J=2.3 Hz), 8.14 (dd, 1H, J=2.3, 8.7 Hz), 7.59 (m, 1H), 7.26 (dd, 2H, J=7.7, 7.9 Hz), 7.16 (d, 1H, J=8.5 Hz), 3.27 (d, 2H, J=5.7 Hz), 2.20 (s, 6H), 0.97 (s, 6H). LC-MS (M+H⁺): 509. Anal. Calcd. for C₂₁H₂₂F₂N₆O₃S₂: C, 49.60; H, 4.36; N, 16.52; S, 12.61. Found: C, 49.78; H, 4.49; N, 16.50; S, 12.81.

The starting materials for above were prepared as follows:
2, N2, N2-trimethyl-propane-1,2-diamine Hydrochloride 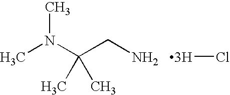
To a suspension of LiAlH₄ (2.71 g, 71.4 mmol) in THF (15 mL) at 0° C. was added a solution of 2-dimethylamino-2-methyl-propionitrile (4.00 g, 35.7 mmol, Yang, D.; Brémont, B.; Shen, S.; Kefi, S.; Langlois, M. Eur. J. Med. Chem. 1996, 31, 231-239) in THF (10 mL). The mixture was stirred at 0° C. for 3 h, diluted with ether (150 mL), and while maintaining cooling with an ice bath, sat aq Na₂CO₃ was carefully added dropwise until no hydrogen evolution was witnessed. Na₂SO₄ was then added, the mixture stirred for 10 min, and filtered through a pad of Celite. HCl gas was bubbled through the filtrate. The resultant white precipitate was washed with ether, quickly transferred into a flask, and dried over vacuum to give 6.47 g (82%) of a white hygroscopic solid, which was stored under argon and used without further purification. ¹H NMR (DMSO-d₆): δ 3.19 (s, 2H), 2.65 (s, 6H), 1.36 (s, 6H).

### Example 1M

### 6-[4-Amino-5-(2,6-difluoro-benzoyl)-thiazol-2-yiamino]-N-2-methylamino-2-methyl-propyl)-nicotinamide

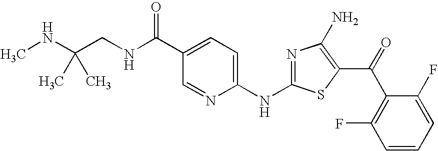

The title compound was prepared in a manner analogous to that used in Example 1A. 6-[4-Amino-5-(2,6-difluoro-benzoyl)-thiazol-2-ylamino]-nicotinic acid (0.376 g, 1.00 mmol) and 2,N2-dimethyl-propane-1,2-diamine hydrochloride (2.12 mmol) provided a yellow solid in 64% yield.

¹H NMR (DMSO-d₆): □ 8.79 (d, 1H, J=2.1 Hz), 8.11 (dd, 1H, J=2.1, 8.7 Hz), 7.93(bs, 2H), 7.57 (m, 1H), 7.20 (dd, 2H, J=7.5, 8.3 Hz), 7.11 (d, 2H, J=8.7 Hz), 3.05 (d, 2H, J=5.8 Hz), 2.04 (s, 3H). LC-MS (M+H⁺): 461. Anal. Calcd. for C₂₁H₂₂F₂N₆O₂S.0.2H₂O: C, 54.35; H, 4.86; N, 18.11; S, 6.91. Found: C, 54.34; H, 4.86; N, 17.88; S, 6.80.

The starting material for above were prepared as follows:
N2,N2-dimethyl-propane-1,2-diamine Hydrochloride

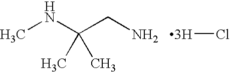

To a suspension of LiAlH₄ (4.62 g, 121.8 mmol) in ether (100 mL) at 0° C. was added a solution of 2-methylamino-2-methyl-propionitrile (5.97 g, 60.9 mmol, the procedure from Yang, D.; Brémont, B.; Shen, S.; Kefi, S.; Langlois, M. Eur. J. Med. Chem. 1996, 31, 231-239) in ether (15 mL). The mixture stirred at 0° C. for 3 h, diluted with ether (150 mL), and while maintaining cooling with an ice bath, sat aq Na₂CO₃ was carefully added dropwise until no hydrogen evolution was witnessed. Na₂SO₄ was then added, the mixture stirred for 10 min, and filtered through a pad of Celite. HCl gas was bubbled through the filtrate. The resultant white precipitate was washed with ether, quickly transferred into a flask, and dried over vacuum to give 7.85 of a white hygroscopic solid, which was stored under argon and used without further purification. ¹H NMR (D20): □ 3.24 (2H, s), 2.63 (3H, s), 1.39 (6H, s).

### Example 2A

### {4-Amino-2-[5-(4-methyl-piperazin-1-yl)-pyridin-2-ylamino]-thiazol-5-yl}-(2,6-difluoro-phenyl)-methanone

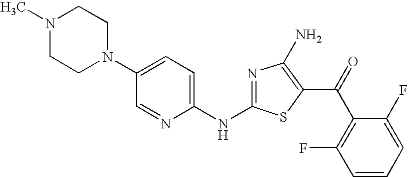

To a solution of 1-(6-isothiocyanato-pyridin-3-yl)-4-methyl-piperazine (241 mg, 1.03 mmol) in CH₃CN (3 mL) was added cyanamide (47 mg, 1.13 mmol) and DBU (0.17 mL, 1.13 mmol). After 0.5 h, 2-bromo-2′,6′-difluoroacetophenone (242 mg, 1.03 mmol) was added. After 0.5 hour, DBU (0.71 mL, 1.13 mmol) was added. After an additional 0.5 h, the solvent was removed, dissolved in MeOH (1 mL) and suspended in water (25 mL). The yellow precipitate was filtered off, washed with water, and recrystallized from EtOH to give 299 mg (67% yield) of an amorphous yellow solid.

¹H NMR (DMSO-d₆): □ 11.75 (s, 1H), 7.92 (d, 1H, J=2.5 Hz), 7.57(m, 1H), 7.49 (dd, 1H, J=2.5, 9.0 Hz), 7.24 (dd, 1H, J=7.7, 7.9 Hz), 7.02 (d, 1H, J=9.0 Hz), 2.22 (s, 3H).

LC-MS (M−H⁺): 429. Anal. Calcd. for C₂₀H₂₀F₂N₆OS.2.0H₂O: C, 51.49; H, 5.19; N, 18.01; S, 6.87. Found: C, 51.66; H, 5.17; N, 17.95; S, 6.83.

The starting materials for above were prepared as follows:
1-Methyl-4-(6-nitro-pyridin-3-yl)-piperazine 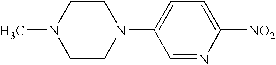
Into a solution of 5′-bromo-2-nitropyridine (2.0 g, 9.85 mmol) in DMSO (10 mL) were added K₂CO₃ (2.72 g, 19.7 mmol), 1-methylpiperazine (1.64 mL, 14.8 mmol), and tetrabutylammonium iodide (36 mg). The mixture was stirred at 120° C. overnight, allowed to cool, and acidified with 1N HCl. The mixture was extracted with CH₂Cl₂, the aqueous layer was basified with sat aq Na₂CO₃, and further extracted with CH₂Cl₂. The combined organic layers were dried over Na₂SO₄ and concentrated to give a wet brown solid, which was washed with small amount of water. The solid dried under vacuum to 2.16 g (99% yield) of yellow powder. ¹H NMR (CDCl₃): δ 8.16 (d, 1H, J=9.1 Hz), 8.14 (d, 1H, J=3.0 Hz), 7.20 (dd, 1H, J=3.0, 9.1 Hz), 3.49 (dd, 4H, J=4.9, 5.3 Hz), 2.61 (dd, 4H, J=4.9, 5.3 Hz), 2.39 (s, 3H).
5-(4-Methyl-piperazin-1-yl)-pyridin-2-ylamine

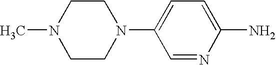

1-Methyl-4-(6-nitro-pyridin-3-yl)-piperazine (1.11 g, 5.02 mmol) and 10% Pd/C (110 mg) in MeOH (15 mL) were stirred for 5 h and filtered. The filtrate was concentrated to give 951 mg (99% yield) of a yellow solid that was used without further purification.

¹H NMR (CDCl₃): δ 7.78 (d, 1H, J=3.0 Hz), 7.18 (dd, 1H, J=3.0, 8.9 Hz), 6.48 (d, 1H, J=8.9 Hz), 3.08 (dd, 4H, J=4.9, 4.9 Hz), 2.61 (dd, 4H, J=4.9, 4.9 Hz), 2.37 (s, 3H).
1-(6-Isothiocyanato-pyridin-3-yl)-4-methyl-piperazine

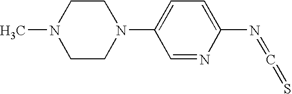

To a solution of 5-(4-methyl-piperazin-1-yl)-pyridin-2-ylamine (400 mg, 2.09 mmol) in CHCl₃ (15 mL) were added sequentially saturated aqueous NaHCO₃ and thiophosgene (0.175 mL, 2.30 mmol). After 30 min, the organic layer was separated, dried over Na₂SO₄, and evaporated to give 441 mg (90%) of yellow solid, which was used without any further purification.

¹H NMR (CDCl₃): δ 7.98 (d, 1H, J=2.8 Hz), 7.10 (dd, 1H, J=2.8, 8.9 Hz), 6.96 (d, 1H, J=8.9 Hz), 3.23 (dd, 4H, J=4.9, 4.9 Hz), 2.58 (dd, 4H, J=4.9, 4.9 Hz), 2.34 (s, 3H).

### Example 3A

### 6-[4-Amino-5-(2,6-difluoro-benzoyl)-thiazol-2-ylamino]-N-2-methylamino-ethyl)-nicotinamide

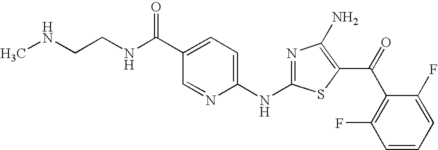

A solution of [2-({6-[4-amino-5-(2,6-difluoro-benzoyl)-thiazol-2-ylamino]-pyridine-3-carbonyl}-amino)-ethyl]-methyl-carbamic acid tert-butyl ester (410 mg, 0.80 mmol) in CH₂Cl₂ (3 mL) was treated with TFA (3 mL). After 30 min at ambient temperature, the solvent was removed in vacuo, the residue was taken up in MeOH (3 mL), and precipitated with water. The suspension was stirred rapidly for 30 min, filtered, the solid was washed with water and dried under vacuum to give a yellow powder in 88% yield.

¹H NMR (DMSO-d₆): □ 8.56 (d, 1H, J=1.7 Hz), 8.20 (dd, 1H, J=5.5, 5.7 Hz), 7.92 (dd, 1H, J=1.7, 8.7 Hz), 7.77 (bs, 2H), 7.39 (m, 1H), 7.05 (dd, 1H, J=7.7, 8.1 Hz), 3.15 (bs, 2H), 2.24 (t, 2H, J=6.3 Hz), 2.12 (s, 3H). LC-MS (M+H⁺): 433. Anal. Calcd. for C₁₉H₁₈F₂N₆O₂S.1.0H₂Oe 0.7 MeOH: C, 50.04; H, 4.86; N, 17.77; S, 6.78. Found: C, 50.01; H, 4.50; N, 17.40; S, 6.64.

The starting material for above were prepared as follows:
[2-({6-[4-Amino-5-(2,6-difluoro-benzoyl)-thiazol-2-ylamino]-pyridine-3-carbonyl}-amino)-ethyl]-methyl-carbamic Acid tert-Butyl Ester 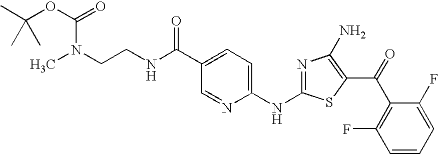
[2-({6-[4-Amino-5-(2,6-difluoro-benzoyl)-thiazol-2-ylamino]-pyridine-3-carbonyl}-amino)-ethyl]-methyl-carbamic acid tert-butyl ester was made in a manner analogous to that used in Example 1A. 6-[4-Amino-5-(2,6-difluoro-benzoyl)-thiazol-2-ylamino]-nicotinic acid (0.376 g, 1.00 mmol; from Example 1A) and (N-2-aminoethyl)-N-methyl carbamic acid t-butyl ester (AstaTech; 192 mg, 1.20 mmol mmol) provided a yellow solid in 80% yield, which was used without any further purification.

LC-MS (M+H⁺): 533.15.

### Comparative Example C1A

### 5-[4-Amino-5-(2,6-difluoro-benzoyl)-thiazol-2-ylamino]-pyridine-2-carboxylic acid (1-methyl-pyrrolidin-2S-ylmethyl)-amide

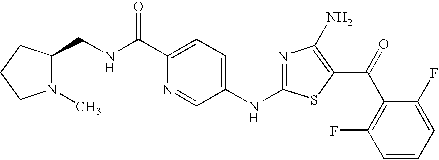

The title compound was prepared in a manner analogous to that for Example 1A: 5-[4-amino-5-(2,6-difluoro-benzoyl)-thiazol-2-ylamino]-pyridine-2-carboxylic acid and (N-methyl-pyrrolidin-2S-yl)-methylamine (0.115 g, 1.01 mmol; Sassaman, M. B.; Giovanelli, J.; Sood, V. K.; Eckelman, W. C. Bioorg. Med. Chem. 1998, 6, 1759-1766) were allowed to couple over 18 h and purified via flash column chromatography with 1% (58% NH₄OH) in 15% MeOH/CHCl₃ as eluant to give a yellow foam in 62% yield. ¹H NMR (CD₃OD): 08.90 (d, 1H, J=2.4 Hz), 8.54 (dd, 1H, J=2.6, 8.6 Hz), 8.18 (d, 1H, J=8.6 Hz), 7.64-7.58 (m, 1H), 7.18 (dd, 2H, J=7.6, 8.3 Hz), 3.78 (dd, 1H, J=3.9, 13.5 Hz), 2.58 (s, 3H). LCMS (M+H⁺): 473. Anal. Calcd. for C₂₂H₂₂F₂N₆O₂S.0.4H₂O.4MeOH: C, 54.62; H, 4.99; N, 17.06; S, 6.51. Found: C, 55.01; H, 5.22; N, 17.05; S, 6.14.

The starting materials for above were prepared as follows:
5-Nitro-pyridine-2-carboxylic Acid 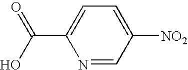
The reported procedure from Oehlke, J.; Schroetter, E.; Dove, S.; Schick, H.; Niedrich, H. Pharmazie 1983, 38, 591-596, was slightly modified. When N,N-dimethylformamide (DMF) was used as the solvent instead of dimethylsulfoxide (DMSO) in the substitution of 2-bromo-5-nitropyridine by copper(I) cyanide, subsequent hydrolysis provided the described acid in an improved 70% yield, typically used without any further purification.
5-Nitro-pyridine-2-carboxylic Acid Ethyl Ester

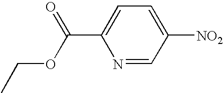

A solution of 5-nitro-pyridine-2-carboxylic acid (38.0 g, 226 mmol) in thionyl chloride (50.0 ml, 686 mmol) was heated at reflux for 1 h and concentrated to a solid, which was dissolved in anhydrous ethanol (100 mL), refluxed 2 h, and allowed to cool. The solvent was evaporated in vacuo. The resultant solid residue was dissolved in ethyl acetate (500 mL), washed with 1 M aq. sodium bicarbonate (2×300 mL), dried over sodium sulfate, and concentrated to give 39.0 g (86% yield) of a yellow solid, which was more than 95% pure by ¹H NMR and LCMS.

¹H NMR (DMSO-d₆): □9.46 (d, 1H, J=2.1 Hz), 8.76 (dd, 1H, J=2.1, 8.6 Hz), 8.29 (d, 1H, J=8.6 Hz), 4.41 (q, 2H, J=7.1 Hz), 1.36 (t, 3H, J=7.1 Hz). LCMS-APCI (M+H⁺): 197.
5-Amino-pyridine-2-carboxylic Acid Ethyl Ester

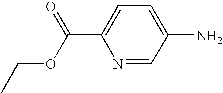

A suspension of 5-nitro-pyridine-2-carboxylic acid ethyl ester (2.00 g, 10.2 mmol) and 10% Pd/C (200 mg) in ethanol (30 mL) was shaken under hydrogen at 40 psi for 4 h. The catalyst was filtered onto Celite and rinsed. The filtrate was concentrated in vacuo to give 1.47 g (87% yield) of a yellow solid, which displayed a ¹H NMR that matched the literature (Min, R. S.; Aksenov, V. S. Chem. Heterocycl. Compd. (Engl. Transl.) 1988, 24, 885-886) and was used without any further purification.
5-Isothiocyanato-pyridine-2-carboxylic Acid Ethyl Ester

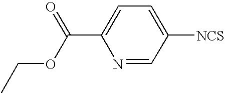

To a suspension of 5-amino-pyridine-2-carboxylic acid ethyl ester (330 mg, 1.98 mmol) in acetone (5 mL) and 25% NaHCO₃ (5 mL) at 0° C. was added a solution of thiophosgene (0.182 mL, 2.38 mmol) in acetone (2 mL). The resulting mixture was allowed to warm to ambient temperature, and acetone was removed in vacuo to give a cream-colored precipitate, which was filtered, washed with H₂O, and dried to give 0.310 g (75% in yield) of a cream-colored powder, which was used without further purification.
5-[4-Amino-5-(2,6-difluoro-benzoyl)-thiazol-2-ylamino]-pyridine-2-carboxylic Acid Ethyl Ester

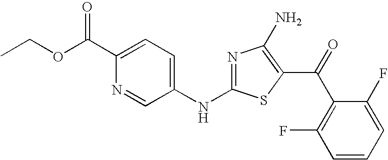

Prepared in a manner analogous to that for 6-[4-amino-5-(2,6-difluoro-benzoyl)-thiazol-2-ylamino]-nicotinic acid ethyl ester from Example 1A: 5-isothiocyanato-pyridine-2-carboxylic acid ethyl ester and 2-bromo-2′,6′-difluoroacetophenone gave a brown solid, which was purified via column chromatography with 1% (58% NH₄OH) in 15% MeOH/CHCl₃ as eluant to give 0.310 g (77% yield) of a yellow solid, which was used without any further purification.

¹H NMR (CD₃OD): □ 8.88 (d, 1H, J=2.5 Hz), 8.50 (dd, 1H, J=2.6, 8.7 Hz), 8.12 (d, 1H, J=8.7 Hz), 7.52-7.44 (m, 1H), 7.10 (dd, 1H, J=7.7, 8.1 Hz), 4.44 (q, 2H, J=7.1 Hz), 1.42 (t, 3H, J=7.1 Hz).
5-[4-Amino-5-(2,6-difluoro-benzoyl)-thiazol-2-ylamino]-pyridine-2-carboxylic Acid

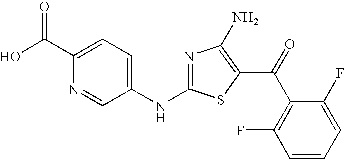

The intermediate was prepared in a manner analogous to 6-[4-amino-5-(2,6-difluoro-benzoyl)-thiazol-2-ylamino]-nicotinic acid from Example 1A. Saponificaton of 5-[4-amino-5-(2,6-difluoro-benzoyl)-thiazol-2-ylamino]-pyridine-2-carboxylic acid ethyl ester provided 0.205 g (76% yield) of a brown solid, which was used without any further purification.

¹H NMR (CD₃OD): □ 8.76 (d, 1H, J=2.6 Hz), 8.45 (dd, 1H, J=2.5, 8.7 Hz), 8.02 (d, 1H, J=8.7 Hz), 7.49-7.25 (m, 1H), 6.99 (dd, 2H, J=7.5, 8.3 Hz).

### Comparative Example C1B

### 5-[4-Amino-5-(2,6-difluoro-benzoyl)-thiazol-2-ylamino]-pyridine-2-carboxylic Acid (2-Dimethylamino-1R-methyl-ethyl)-amide

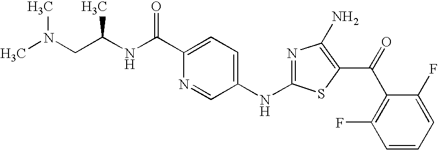

The title compound was prepared in the same manner as Example 1A, 5-[4-amino-5-(2,6-difluoro-benzoyl)-thiazol-2-ylamino]-pyridine-2-carboxylic acid (from Example C1A and 1-dimethylamino-prop-2R-yl-amine bis-toluenesulfonic acid salt (from Example 1I) and subsequent purification via flash column chromatography with 1% (58% NH₄OH)/15% MeOH/CHCl₃ as eluant gave a yellow foam in 55% yield. ¹H NMR (CD₃OD): □9.00 (d, 1H, J=2.5 Hz), 8.67 (dd, 1H, J=2.5, 8.9 Hz), 8.28 (d, 1H, J=8.9 Hz), 7.69 (ddd, 1H, J=6.2, 8.1, 15.3 Hz), 7.30 (dd, 2H, J=7.9, 7.9 Hz), 4.55 (m, 1H), 2.91 (m, 1H), 2.57 (m, 1H), 2.57 (s, 6H), 1.49 (d, 3H, J=6.6 Hz).

LC-ESIMS (M+H⁺): 461.15. Anal. Calcd. for C₂₁H₂₂F₂N₆O₂S.0.5H₂O: C, 53.72; H, 4.94; N, 17.90; S, 6.83. Found. C, 53.44; H, 4.98; N, 17.73; S, 6.67.

Inhibition of Cell Growth: Assessment of Cytotoxicity:

Inhibition of cell growth was measured using the tetrazolium salt assay, which is based on the ability of viable cells to reduce 3-(4,5-dimethylthiazol-2-yl)-2,5-[2H]-diphenyltetrazolium bromide (MTT) to formazan (Mosmann, T. J. Immunol. Methods 1983, 65, 55-63). The water-insoluble purple formazan product was then detected spectrophotometrically. HCT-116 cells were grown in 96-well plates. Cells were plated in the appropriate medium at a volume of 135 μl/well in either McCoy's 5A Medium. Plates were incubated for 4 hours before addition of inhibitor compounds. Different concentrations of inhibitor compounds were added in 0.5% (v/v) dimethylsulfoxide (15 μL/well), and cells were incubated at 37° C. (5% CO₂) for four to six days (depending on cell type). At the end of the incubation, MTT was added to a final concentration of 0.2 mg/mL, and cells were incubated for 4 hours more at 37° C. After centrifugation of the plates and removal of medium, the absorbance of the formazan (solubilized in dimethylsulfoxide) was measured at 540 nm. The concentration of inhibitor compound causing 50%(IC₅₀) or 90%(IC₉₀) inhibition of growth was determined from the linear portion of a semi-log plot of inhibitor concentration versus percentage inhibition. All results were compared to control cells treated only with 0.5% (v/v) dimethylsulfoxide. The IC₅₀ and IC₉₀ values are shown in Table 1.

Inhibition of CDK4/Cyclin D Retinoblastoma Kinase Activity:

A complex of human CDK4 and genetically truncated (1-264) cyclin D3 was purified using traditional biochemical chromatographic techniques from insect cells that had been co-infected with the corresponding baculovirus expression vectors (see e.g., Meijer, L.; Kim, S.-H. In Methods Enzymol. 1997, 283, 113-128). The enzyme complex (5 nM) was assayed with 0.3-0.5 μg of purified recombinant retinoblastoma protein fragment (Rb) as a substrate. The engineered Rb fragment (residues 386-928 of the native retinoblastoma protein; 62.3 kDa) contains the majority of the phosphorylation sites found in the native 106-kDa protein, as well as a tag of six histidine residues for ease of purification. Phosphorylated Rb substrate was captured by microfiltration on a nitrocellulose membrane and quantified using a phosphorimager as described above. For measurement of tight-binding inhibitors, the assay duration was extended to 60 minutes, during which the time dependence of product formation was linear and initial rate conditions were met. K, values were measured as described above and shown in Table 1.

Inhibition of CDK2/Cyclin A Retinoblastoma Kinase Activity:

CDK2 was purified using published methodology (Rosenblatt, J.; De Bondt, H.; Jancarik, J.; Morgan, D. O.; Kim, S. H. J. Mol. Biol. 1993, 230, 1317-1319) from insect cells that had been infected with a baculovirus expression vector. Cyclin A was purified from E. coli cells expressing full-length recombinant cyclin A, and a truncated cyclin A construct was generated by limited proteolysis and purified as described previously (Jeffrey, P. D.; Russo, A. A.; Polyak, K.; Gibbs, E.; Hurwitz, J.; Massague, J.; Pavletich, N. P. Nature 1995, 376, 313-320.). A complex of CDK2 and proteolyzed cyclin A was prepared and purified by gel filtration. The substrate for this assay was the same Rb substrate fragment used for the CDK4 assays, and the methodology of the CDK2/delta cyclin A and the CDK4/delta cyclin D3 assays was essentially the same, except that CDK2 was present at 10 nM or 19 nM. The duration of the assay was 60 or 75 minutes, during which the time dependence of product formation was linear and initial rate conditions were met. K_{I} values were measured as described above and shown in Table 1.

TABLE 1

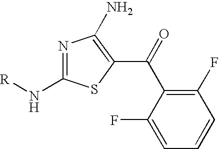

| Example | R | HCT116 IC50, 90 (μM) | Ki CDK4/D (nM) | Ki CDK2/A (nM) |
|---|---|---|---|---|
| 1A | 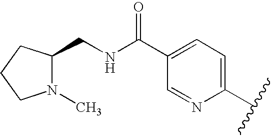 | 0.007, 0.17 | 0.77 | 7.3 |
| 1B | 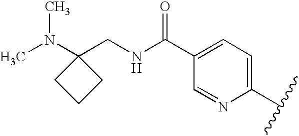 | 0.0058, 0.02 | 2.7 | 39 |
| 1C | 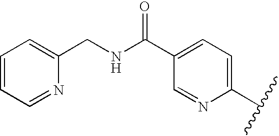 | 0.079, 0.21 | 12 | 7.2 |
| 1D | 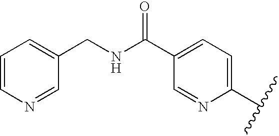 | 0.41, >5 | 6.6 | 5.7 |
| 1E | 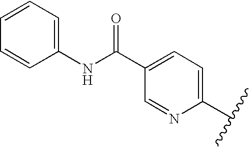 | 0.18, 0.40 | 18 | 17 |
| 1F | 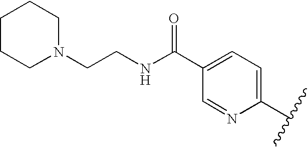 | 0.017, 0.04 | 4.3 | 18 |
| 1G | 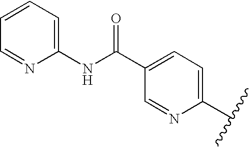 | 0.17, 0.36 | 35 | 18 |
| 1H | 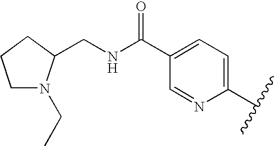 | 0.032, 0.06 | 7.1 | 43 |
| 1I | 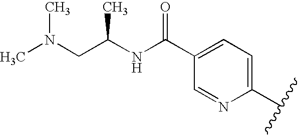 | 0.0026, 0.0057 | 0.61 | 4.4 |
| 1J | 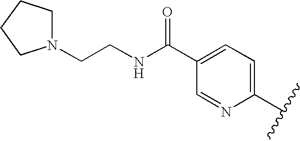 | 0.017, 0.05 | 2.2 | 11 |
| 1K | 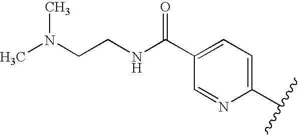 | 0.0088, 0.021 | 1.4 | 9.8 |
| 1L | 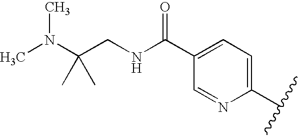 | 0.0025, 0.0085 | 1.3 | 8.6 |
| 1M | 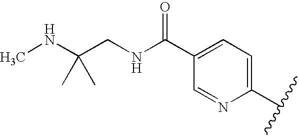 | 0.070, 0.25 | 2.3 | 35 |
| 2A | 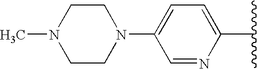 | 0.07, 0.18 | 1.1 | 32 |
| 3A | 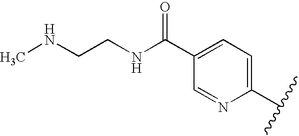 | 0.210, 0.700 | 3.3 | 21 |
| C1A | 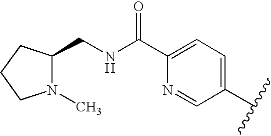 | 0.23, 0.65 | 37 | 320 |
| C1B | 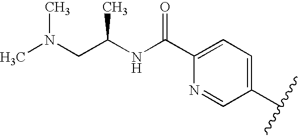 | 0.12, 0.28 | 120 | 400 |

1. A compound represented by the Formula I: 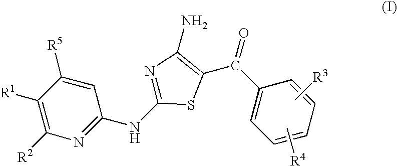
wherein:
R¹ is hydrogen, or an alkenyl, alkynyl, C₁-C₈ alkylamino, aryl, cycloalkyl, carboxamide, sulfonamide or alkoxy group, unsubstituted or substituted with one or more substituents independently selected from the group consisting of alkyl, heteroalkyl, haloalkyl, haloaryl, halocycloalkyl, haloheterocycloalkyl, aryl, cycloalkyl, heterocycloalkyl, heteroaryl, —NO₂, —NH₂, —N—(R_{c})OR_{d}, —CN, —(CH₂), —CN where z is 0-4, halo, —OH, —O—Rₐ, —OR_{b}, —CO—R_{c}, —O—CO—R_{c}, —CO—OR_{c}, —O—CO—OR_{c}, —O—CO—O—CO—R_{c}, —O—OR_{c}, keto (═O), thioketo (═S), —SO₂—R_{c}, —SO—R_{c}, —NR_{d}Rₑ, —CO—NR_{d}Rₑ, —O—CO—NR_{d}Rₑ, —NR_{c}—CO—NR_{d}Rₑ, —NR_{c}—CO—Rₑ, NR_{c}C—CO₂ORₑ, —CO—NR_{c}—CO—R_{d}, —O—SO₂—R_{c}, —O—SO—R_{c}, —O—S—R_{c}, —S—CO—R_{c}, —SO—CO—OR_{c}, —SO₂—CO—OR_{c}, —O—SO₃H, —NR_{c}—SR_{d}, —NR_{c}—SO—R_{d}, —NR_{c}—SO₂—R_{d}, —CO—SR_{c}, —CO—SO—R_{c}, —CO—SO₂—R_{c}, —CS—R_{c}, —CSO—R_{c}, —CSO₂—R_{c}, —NR_{c}—CS—R_{d}, —O—CS—R_{c}, —O—CSO—R_{c}, —O—CSO₂—R_{c}, —SO₂—NR_{d}Rₑ, —SO—NR_{d}Rₑ, —S—NR_{d}Rₑ, —NR_{d}—CSO₂—R_{d}, —NR_{c}—CSO—R_{d}, —NR_{c}—CS—R_{d}, —SH, —S—R_{b}, and —PO₂—OR_{c}, where Rₐ is selected from the group consisting of alkyl, heteroalkyl, alkenyl, and alkynyl; Rb is selected from the group consisting of alkyl, heteroalkyl, haloalkyl, alkenyl, alkynyl, halo, —CO—R_{c}, —CO—OR_{c}, —O—CO—O—R_{c}, —O—CO—R_{c}, —NR_{c}—CO—R_{d}, —CO—NR_{d}Rₑ, —OH, aryl, heteroaryl, heterocycloalkyl, and cycloalkyl; R_{c}, R_{d} and Rₑ are each independently selected from the group consisting of hydro, halo, alkyl, heteroalkyl, haloalkyl, alkenyl, alkynyl, —COR_{f}, —COOR_{f}, —O—CO—O—R_{f}, —O—CO—R_{f}, —OH, aryl, heteroaryl, cycloalkyl, and heterocycloalkyl, or R_{d} and Rₑ cyclize to form a heteroaryl or heterocycloalkyl group;
and R_{f} is selected from the group consisting of hydro, alkyl, and heteroalkyl; and where any of the alkyl, heteroalkyl, alkenyl, aryl, cycloalkyl, heterocycloalkyl, or heteroaryl moieties present in the above substituents may be further substituted with one or more additional substituents independently selected from the group consisting of —NO₂, —NH₂, —CN, —(CH₂), —CN where z is 0-4, halo, haloalkyl, haloaryl, —OH, keto, —N(R_{c})OR_{d}, —NR_{d}Rₑ, —CO—NR_{d}Rₑ, —CO—OR_{c}, —CO—R_{c}, —NR_{c}—CO—NR_{d}Rₑ, —C—CO—OR_{c}, —NR_{c}—CO—R_{d}, —O—CO—O—R_{c}, —O—CO—NR_{d}Rₑ, —SH, —O—R_{b}, —O—Rₐ—O—R_{b}, —S—R_{b}, unsubstituted alkyl, unsubstituted aryl, unsubstituted cycloalkyl, unsubstituted heterocycloalkyl, and unsubstituted heteroaryl, where Rₐ, R_{b}, R_{c}, R_{d}, and Rₑ are as defined above;
R² and R⁵ are each independently hydro, halo, C₁₋₂ alkyl, —OCH₃, —OH, —NH₂, —NHCH₃, —N(CH₃)₂, —NO₂, —SH, —SCH₃, —S(O)CH₃, —SO₂CH₃, —P(CH₃)₂, or —PO₃H₂; and
R³ and R⁴ are each independently hydro, halo, methoxyl, or C₁₋₂ alkyl;
or a pharmaceutically acceptable salt.

2. The compound or pharmaceutically acceptable salt according to claim 1, wherein R¹ is an unsubstituted or substituted carboxamide, heterocycloalkyl or sulfonamide group.

3. The compound or pharmaceutically acceptable salt according to claim 1, wherein R² and R⁵ are hydro.

4. The compound or pharmaceutically acceptable salt according to claim 1, wherein R³ and R⁴ are both halo positioned ortho relative to the point of attachment to the carbonyl.

5. The compound or pharmaceutically acceptable salt according to claim 4, wherein R³ and R⁴ are fluoro.

6. A compound represented by the Formula II: 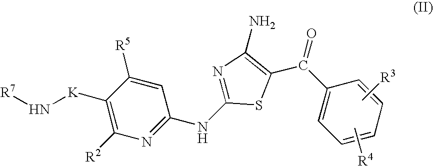
wherein
K is —C(O)— or —SO₂—;
R² and R⁵ are each independently hydro, halo, C₁₋₂ alkyl, —OCH₃, —OH, —NH₂, —NHCH₃, —N(CH₃)₂, —NO₂, —SH, —SCH₃, —S(O)CH₃, —SO₂CH₃, P(CH₃)₂, or PO₃H₂;
R³ and R⁴ are each independently hydro, halo, methoxyl, or C₁₋₂ alkyl;
R⁷ is a C₁-₈ alkyl, C₁₋₈ alkylamino, aryl, C₁₋₈ alkyl-aryl, heteroaryl, heterocycloalkyl, C₁-₈-alkyl-heteroaryl, or C₁-₈ alkyl-heterocycloalkyl, unsubstituted or substituted with C₁-₈ alkyl, halo, methoxyl, aryl, or C₁₋₈ alkyl-aryl;
or a pharmaceutically acceptable salt, prodrug, active metabolite, multimeric form or solvate, or a pharmaceutically acceptable salt of said active metabolite thereof.

7. A compound selected from the group consisting of:

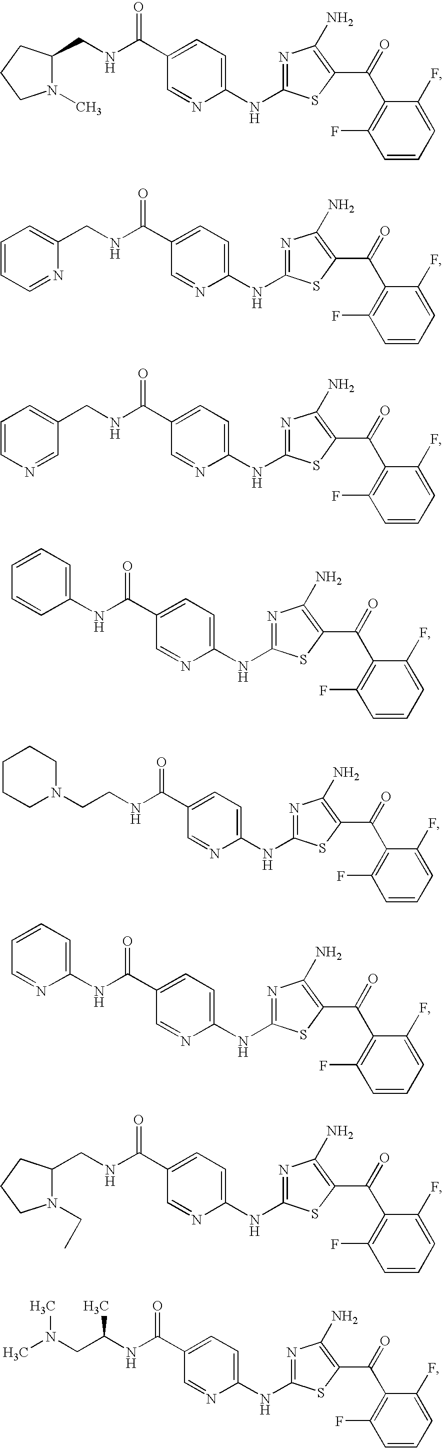

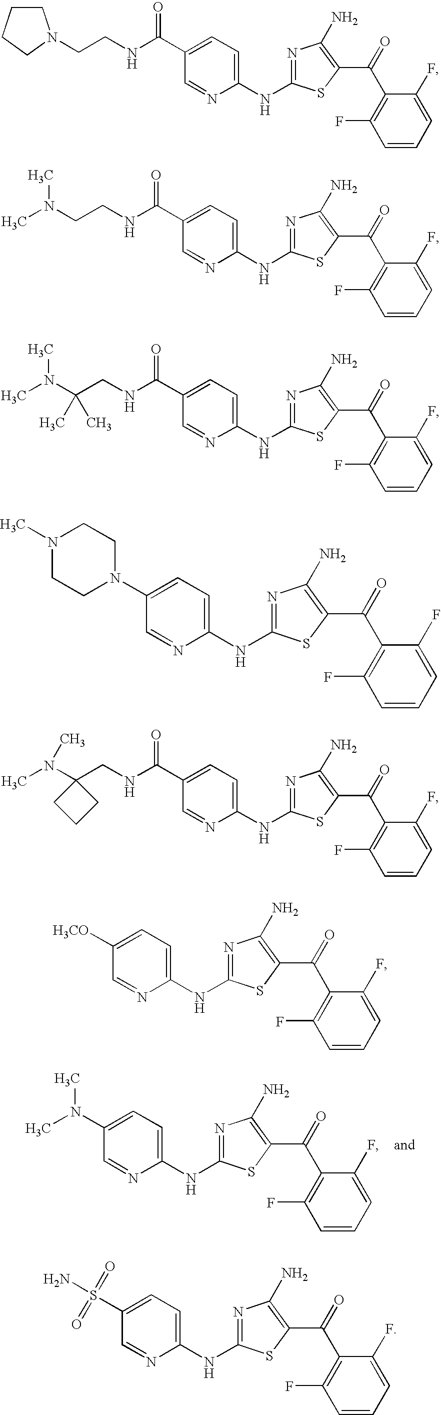

8-15. (canceled)
